(19) United States
(12) Patent Application Publication
(10) Pub. No.: US 2006/0008532 A1
(43) Pub. Date: Jan. 12, 2006

(54) Complexes of protein crystals and ionic polymers

(76) Inventors: Chandrika Govardhan, Lexington, MA (US); Nazer Khalaf, Worcester, MA (US)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 11/169,956

(22) Filed: Jun. 28, 2005

Related U.S. Application Data
(63) Continuation of application No. PCT/US03/41691, filed on Dec. 31, 2003.
(60) Provisional application No. 60/437,775, filed on Dec. 31, 2002.

(51) Int. Cl. A61K 39/395; A61K 38/54; A61K 9/14; A61K 38/19; A61K 38/18; A61K 38/20; A61K 38/22

(52) U.S. Cl. 424/489; 424/85.1; 424/85.2; 424/144.1; 514/12; 424/94.2

(57) ABSTRACT

The present invention relates to complexes of protein crystals and ionic polymers and compositions comprising such complexes. The invention further provides methods for producing these complexes and compositions. The invention further provides methods for treatment of an individual having a disease requiring or ameliorated by sustained release of protein-based therapies.

### TECHNICAL FIELD OF THE INVENTION

The present invention relates to complexes of protein crystals and ionic polymers and compositions comprising them, including sustained release compositions. In addition, the invention provides methods for producing these complexes and compositions. The complexes and compositions of the present invention are particularly useful in the treatment of disease states amenable to treatment by sustained release of protein-based therapies.

### BACKGROUND OF THE INVENTION

Major drawbacks exist in the development of protein therapeutics targeted to various disease states. Commercially non-viable formulations, short in vivo half-lives and negligible oral bioavailability are examples of some of these drawbacks. To date, short in vivo half-lives have limited the development of sustained release protein formulations, which are typically delivered by intraveneous or subcutaneous administration.

To provide sustained release formulations of human growth hormone (hGH), for example, technologies that incorporate hydrogels [Katakam et al., J. Controlled Release, 49(1), 21-26 (1997)], liposomes, oil emulsions, biodegradable polymer microspheres, as well as polyethylene glycol modification [Ross et al., J. Biol. Chem., 271(36), 21696-21977 (1996)] have been developed. However, the resulting formulations display a burst release of the drug, use harsh manufacturing conditions and/or may be complicated to manufacture. This is especially true of hGH formulations based on DL-lactic co-glycolic acid (PLGA) microsphere technology, because the process used to produce the microspheres tends to employ conditions such as elevated temperatures, surfactants, organic solvents and aqueous/organic solvent interface, all of which cause protein denaturation [Herberger et al., Proc. Intl. Symp. Controlled Release of Bioactive Materials, 23, 835-836 (1996); Kim et al., Intl. J. Pharmaceutics, 229(1-2), 107-116 (2001)].

An alternate approach to developing sustained release protein formulations involves the use of crystallized proteins as part of the formulation. For example, crystallized insulin complexed with zinc and protamine exhibits extended release behavior [Krayenbuhl and Rosenberg, Rep. Steno. Mem. Hosp. Nord. Insulinlab. 1: 60-73 (1946)]. Because different crystals can modulate the degree of solubility and the rate of dissolution of a given protein crystal, the development of sustained release formulations based on different crystals having various solubility profiles is desired.

### SUMMARY OF THE INVENTION

In order to develop sustained release compositions of different protein crystals, the present invention exploits the amphoteric nature of proteins. For example, the number of basic and acidic residues available on a protein chain, as well as the pH environment, will determine a protein's overall net charge. Thus, inherent to every protein is an isoelectric pH value (pI) or a specific pH where the net charge of the protein is zero—a pH where protein solubility in water is the lowest and crystallization is most likely to occur. The complexation of polycations to proteins having a low pI (number of acidic groups in a protein exceeds the number of basic groups) or polyanions to proteins having a high pI (number of basic groups in a protein exceeds the number of acidic groups) can result in the protein having advantageous physical properties, including favorable dissolution behavior. Proteins can be crystallized mostly at either their pI or very close to the pI values. However, addition of polyanions or polycations to protein crystals at a pH of solution near the isoelectric point of the protein may result in poor complexation.

Such drawbacks of conventional complexation techniques may be avoided by using the methods of the present invention. Advantageously, physiologically-compatible sustained release complexes of protein crystals and ionic polymers and compositions comprising them are obtained. To that end, the invention provides methods for preparing such complexes and compositions and for the treatment of disease states requiring or ameliorated by sustained release of drug therapies.

### BRIEF DESCRIPTION OF DRAWINGS

FIG. 1 illustrates hGH crystals grown in the presence of 85 mM calcium acetate and 100 mM Tris-HCl (pH 8.6) and Protamine sulfate (1 mg/ml) as imaged by optical microscopy. See Example 4.

FIG. 2 shows solubility of ammonium phosphate, sodium citrate, dibasic sodium phosphate and calcium acetate/Protamine salts of hGH monitored at 280 nm as a function of time. See Example 5.

FIG. 3 illustrates hGH crystals grown in the presence of 85 mM calcium acetate, 6% (v/v) PEG-6000, 100 mM Tris-HCl (pH 8.6) and Protamine sulfate (1 mg/ml) as imaged by optical microscopy. See Example 9.

FIG. 4 shows solubility of hGH crystals grown according to Examples 6-10 monitored at 280 nm as a function of time in minutes. See Example 11.

FIG. 5 illustrates the dissolution characteristics of hGH crystals (formed in the presence of 85 mM calcium acetate, 2% (v/v) PEG-6000 and 100 mM Tris-HCl (pH 8.6)) upon the addition of varying amounts of Protamine sulfate. Protamine sulfate was added to the hGH crystals and allowed to sit for 1 hour before the concentration of soluble hGH in the supernatant was measured by RP-HPLC (Area). See Example 12.

FIG. 6 illustrates rasburicase crystals grown in the presence of 5% ethanol and 15% PEG-6000 (pH 8.5) as imaged by optical microscopy. See Example 14.

FIG. 7 illustrates the percent cumulative dissolution of Rasburicase either bare or complexed with ionic polymers, i.e., polyarginine, polylysine, protamine and polyorthinine. See Example 18.

FIG. 8 illustrates oxalate oxidase crystals grown in the presence of 40% PEG-600 in 100 mM phosphate citrate buffer (pH 4.2) as imaged by optical microscopy. See Example 19.

FIG. 9A shows the concentration of hGH in blood serum as a function of time for female juvenile cynomologous monkeys subcutaneously administered daily soluble hGH (Group 1), sodium crystals of hGH complexed with polyarginine (Group 2) and sodium crystals of hGH complexed with protamine (Group 3) according to Table 6. See Example 26.

FIG. 9B shows the concentration of IGF-1 in blood serum as a function of time for female juvenile cynomologous monkeys subcutaneously administered daily soluble hGH (Group 1), sodium crystals of hGH complexed with polyarginine (Group 2) and sodium crystals of hGH complexed with protamine (Group 3) according to Table 8. See Example 26.

FIG. 10A illustrates the seven-day growth of male Wistar rats that had been subcutaneously administered control (Group 1, once daily over seven days), soluble hGH (Groups 4 and 5, once daily over seven days) and crystalline hGH (Groups 6, 7, 9 and 10, once over seven days) according to Table 10. See Example 27. Note that monkey dose refers to high dose, i.e., 5.6 mg/kg/week.

FIG. 10B illustrates the daily induced weight gain (grams) over a seven day period for male Wistar rats that had been subcutaneously administered control (Group 1, once daily over seven days), soluble hGH (Groups 4 and 5, once daily over seven days and crystalline hGH (Groups 6, 7, 9 and 10, once over seven days) according to Table 11. See Example 27.

### DETAILED DESCRIPTION OF THE INVENTION

Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, column chromatography, optical microscopy, UV-VIS spectroscopy, pharmokinetic analyses, recombinant DNA methods, peptide and protein chemistries, nucleic acid chemistry and molecular biology described herein are those well known and commonly used in the art.

The following terms, unless otherwise indicated, shall be understood to have the following meanings:

The term “complex” refers to a crystal of a protein and an ionic compound. Alternatively, the term complex can refer to a crystal of a protein, an ionic compound and an excipient.

The term “protein crystal” refers to one form of the solid state of matter having a three-dimensional crystal lattice, which is distinct from the amorphous solid state. Whether a protein is in a crystalline state may be determined by any method known in the art, e.g., X-ray diffraction or powder X-ray diffraction.

The term “amorphous solid” or “amorphous precipitate” is a non-crystalline solid form of a protein, which has no three-dimensional crystal lattice structure characteristic of the crystalline solid state.

The term “spherical protein particle (SPP)” is a protein composite that has a sphere radius on the order of nanometers. The composite contains crystalline protein in combination with one or more pharmaceutically or diagnostically acceptable ingredients or excipients.

The term “ionic compound” refers to any polymer (homopolymer or heteropolymer) or small molecule, including peptides, that contain at least two charged groups and a net charge of at least 2 under a given pH environment. The term ionic compound also includes polyelectrolytes.

The term “therapeutic protein” refers to a protein which is administered to a living organism in a formulation or composition or a pharmaceutical formulation or composition. Examples of therapeutic proteins or prophylactic proteins include hormones glucagons, such as glucagon-like peptide 1 and parathyroid hormone, antibodies, fusion proteins, Enbrel (etanercept) (Note that Enbrel is a dimeric fusion protein consisting of the extracellular ligand-binding portion of the human 75 kilodalton (p75) tumor necrosis factor receptor (TNFR) linked to the Fc portion of human IgG1. The Fc component of etanercept contains the CH2 domain, the CH3 domain and hinge region, but not the CH1 domain of IgG1), inhibitors, growth factors, nerve growth hormones, blood clotting factors (e.g., Factor IX), adhesion molecules, bone morphogenic proteins and lectins trophic factors, cytokines such as TGF-β, IL-2, IL-4, α-IFN, β-IFN, γ-IFN, TNF, IL-6, IL-8, lymphotoxin, IL-5, Migration inhibition factor, GMCSF, IL-7, IL-3, monocyte-macrophage colony stimulating factors, granulocyte colony stimulating factors (e.g., CSF-3), multidrug resistance proteins, other lymphokines, toxoids, erythropoietin, Factor VIII, amylin, TPA, dornase-α, α-1-antitrypsin, human growth hormones, nerve growth hormones, bone morphogenic proteins, urease, toxoids, fertility hormones, FSH, LSH, Alteplase and tissue plasminogen activator (TPA).

Therapeutic proteins, such as the following, are also included:
leukocyte markers, such as CD2, CD3, CD4, CD5, CD6, CD7, CD8, CD11a, CD11b, CD11c, CD13, CD14, CD18, CD19, CE20, CD22, CD23, CD27 and its ligand, CD28 and its ligands B7.1, B7.2, B7.3, CD29 and its ligands, CD30 and its ligand, CD40 and its ligand gp39, CD44, CD45 and isoforms, Cdw52 (Campath antigen), CD56, CD58, CD69, CD72, CTLA-4, LFA-1 and TCR;
histocompatibility antigens, such as MHC class I or II antigens, the Lewis Y antigens, SLex, SLey, SLea and SLeb;
integrins, such as VLA-1, αIIβ, βIIIα VLA-2, VLA-3, VLA-4, VLA-5, VLA-6 and LFA-1;
adhesion molecules, such as Mac-1 and p150,95;
selectins, such as L-selectin, P-selectin and E-selectin and their counterreceptors VCAM-1, ICAM-1, ICAM-2 and LFA-3;
interleukins, such as IL-1, IL-2, IL-3, IL-4, IL-5, IL-6, IL-7, IL-8, IL-10, IL-11, IL-12, IL-13, IL-14 and IL-15;
interleukin receptors, such as IL-1R, IL-2R, IL-4R, IL-5R, IL-6R, IL-7R, IL-8R, IL-10R, IL-11R, IL-12R, IL-13R, IL-14R, IL-15R, IL-1 trap, IL-4 trap, IL-6 trap and IL-13 trap;
chemokines, such as PF4, RANTES, MIP1α, MCP1, NAP-2, Groα, Groβ and IL-8;
growth factors, such as TNFalpha, TGFbeta, BMPs, GDFs, neuregulins, TSH, VEGF/VPF, PTHrP, EGF family, EGF, PDGF family, endothelin, pegvisomant and gastrin releasing peptide (GRP);
growth factor receptors, such as TNFalphaR, RGFbetaR, TSHR, VEGFR/VPFR, VEGF trap, FGFR, EGFR, PTHrPR, PDGFR family, EPO-R, GCSF-R, recombinant human soluble p55 TNF receptor (TBP-1 protein) and other hematopoietic receptors;
interferon receptors, such as IFNαR, IFNβR and IFNγR;
fusion proteins;
Igs and their receptors, such as IgE, FceRI and FceR11; and
blood factors, such as complement C3b, complement C5a, complement C5b-9, Rh factor, fibrinogen, fibrin and myelin associated growth inhibitor.

The term “glycoprotein” is defined as a molecule comprising a carbohydrate moiety and a proteinaceous moiety.

The protein constituent of the complexes and compositions of this invention may be any natural, synthetic or recombinant protein antigen including, for example, tetanus toxoid, diptheria toxoid, viral surface proteins, such as CMV glycoproteins B, H and gCIII, HIV-1 envelope glycoproteins, RSV envelope glycoproteins, HSV envelope glycoproteins, EBV envelope glycoproteins, VZV envelope glycoproteins, HPV envelope glycoproteins, Influenza virus glycoproteins, Hepatitis family surface antigens; viral structural proteins, viral enzymes, parasite proteins, parasite glycoproteins, parasite enzymes and bacterial proteins.

Also included are tumor antigens, such as her2-neu, mucin, CEA and endosialin. Allergens, such as house dust mite antigen, lol p1 (grass) antigens and urushiol are included.

Toxins, such as pseudomonas endotoxin and osteopontin/uropontin, snake venom and bee venom are included.

Also included are glycoprotein tumor-associated antigens, for example, carcinoembryonic antigen (CEA), human mucins, her-2/neu and prostate-specific antigen (PSA) [R. A. Henderson and O. J. Finn, Advances in Immunology, 62, pp. 217-56 (1996)].

The term polymer refers to a molecule having a molecular weight of approximately 5,000 or greater, which is composed of two or more monomer units of less than 5,000 molecular weight covalently bonded together. According to an alternate embodiment of this invention, the polymer can be comprised of two or more monomers, including dimers, trimers, tetramers and so on. A polymer can be a homopolymer or heteropolymer, including copolymers.

The term copolymer comprises a polymer having two or more different monomer units per chain. The sequence of monomer units within the overall composition of a copolymer can be alternating, block, or statistical [Odian, Principles of Polymerization, 3^{rd} Ed., 142-149 (1991)].

A polypeptide is defined as a chain of greater than 50 amino acids and/or imino acids connected to one another. An oligopeptide is defined as two to 50 amino acids and/or imino acids connected to one another.

A protein is a large macromolecule having a molecular weight of greater than 2,000 and is composed of one or more polypeptide chains.

The term “dendrimer” refers to a dendritic macromolecule, which is a synthetic 3-dimensional macromolecule prepared in a step-wise fashion from simple branched monomer units, the nature and functionality of which can be easily controlled and varied. The unique architecture and monodisperse structure of a dendrimer has been shown to result in significantly improved physical and chemical properties when compared to traditional linear polymers. As a consequence, dendrimers are now considered to be one of the prime nanometer-scale building blocks for advanced drug-delivery systems.

Dendrimers are similar to ordinary organic molecules for the first three generations. They are small and without consistent or specific three-dimensional structure. By the fourth generation, dendrimers start to become spherical and to take on a preferred three-dimensional structure. By the fifth generation, dendrimers have a consistent and specific three dimensional structure and beyond the fifth generation, dedrimers become highly structured spheres. One embodiment of the present invention relates to dendrimers that are at least two generations. In another embodiment of the present invention, the dendrimers can be either positively or negatively charged.

The term “polycation” refers to an oligomer (at least two monomer units) or polymer chain that has a net positive charge under an appropriate pH environment. Examples of polycations include Protamine, polyarginine, polylysine, polyhistidine, histones, myelin basic protein, polymyxin B sulfate, dodecyltrimethylammonium bromide, bradykinin, spermine, putrescine, octylarginine and synthetic peptides and dendrimers.

The term “polyanion” refers to an oligomer (at least two monomer units) or polymer chain that has a net negative charge under an appropriate pH environment. Examples of polyanions include polyglutamate, polyaspartate, polyacrylate, polycyanoacrylates, polylactate, poly-B-hydroxybutyrate, polyvinylpyrollidone, hyaluronic acid, heparin, sulfated polysaccharides, dextran sulfates, heparin sulfates, polyposphates and dendrimers.

The term “suspension” refers to an insoluble phase dispersed within a soluble phase.

Isotonic solutions have the same osmotic pressure as human physiological fluids. An “isotonicity agent” is any molecule or compound that can be used to adjust osmotic pressure in a given fluid.

Complexes and compositions of the instant invention can be combined with any pharmaceutically acceptable excipient. According to this invention, a “pharmaceutically acceptable excipient” is an excipient that acts as a filler or a combination of fillers used in pharmaceutical compositions. Preferred excipients include: 1) amino acids such as glycine, arginine, aspartic acid, glutamic acid, lysine, asparagine, glutamine, proline; 2) carbohydrates, e.g., monosaccharides such as glucose, fructose, galactose, mannose, arabinose, xylose, ribose; disaccharides, such as lactose, trehalose, maltose, sucrose; polysaccharides, such as maltodextrins, dextrans, starch, glycogen; alditols, such as mannitol, xylitol, lactitol, sorbitol; and 3) glucuronic acid and galacturonic acid. Other excipients include cyclodextrins, such as methyl cyclodextrin, hydroxypropyl-β-cyclodextrin and the like; inorganic salts, such as sodium chloride, potassium chloride, magnesium chloride, phosphates of sodium and potassium, boric acid, ammonium carbonate and ammonium phosphate; organic salts, such as acetates, citrate, ascorbate, lactate; emulsifying or solubilizing/stabilizing agents like acacia, diethanolamine, glyceryl monostearate, lecithin, monoethanolamine, oleic acid, oleyl alcohol, poloxamer, polysorbates, sodium lauryl sulfate, stearic acid, sorbitan monolaurate, sorbitan monostearate, and sorbitan derivatives, polyoxyl derivatives, wax, polyoxyethylene derivatives, sorbitan derivatives; and viscosity increasing reagents, such as agar, alginic acid and its salts, guar gum, pectin, polyvinyl alcohol, polyethylene oxide, cellulose and its derivatives propylene carbonate, polyethylene glycol, hexylene glycol, tyloxapol. Salts of any of the foregoing compounds may also be used. A further preferred group of excipients includes sucrose, trehalose, lactose, sorbitol, lactitol, inositol, salts of sodium and potassium such as acetate, phosphates, citrates, borate, glycine, arginine, polyethylene oxide, polyvinyl alcohol, polyethylene glycol, hexylene glycol, methoxy polyethylene glycol, gelatin, hydroxypropyl-β-cyclodextrin, polylysine, polyarginine.

In one embodiment of this invention, the excipient is selected from the group consisting of: salts, alcohols, carbohydrates, proteins, lipids, surfactants, polymers and polyamino acids. In a another embodiment, the excipient is selected from the group consisting of: detergents, pluronic polyols, polyols, glycoaminoglycans, amino acids, starch, glycerol, monosaccharides, disaccharides, cellulose, providone dextrin, polysorbates, hydroxypropyl cellulose and ascorbic acid.

Complexes and compositions according to this invention can also be combined with a carrier or adjuvant, a substance that, when added to a therapeutic, speeds or improves its action. Examples of adjuvants include, for example, Freud's adjuvant, ion exchanges, alumina, aluminum stearate, lecithin, buffer substances, such as phosphates, glycine, sorbic acid and potassium sorbate, partial glyceride mixtures of saturated vegetable fatty acids, waters, salts or electrolytes, such as Protamine sulfate, disodium hydrogen phosphate, sodium chloride, zinc salts, colloidal silica, magnesium, trisilicate, celluslose-based substances and polyethylene glycol. Adjuvants for gel base forms may include, for example, sodium carboxymethylcelluslose, polyacrylates, polyoxyethylene-polyoxypropylene-block copolymers, polyethylene glycol and wood wax alcohols.

Complexes and compositions of this invention can also be combined with stabilizers. In one embodiment of the invention, the stabilizer is selected from the group consisting of: sugars, polyols, amino acids, soluble proteins and detergents.

The term “crystallization reagent mix” is defined as a composition which includes a salt, PEG, buffer and other ingredients needed for protein or polymer crystallization.

One embodiment of this invention relates to a complex comprising protein crystals and ionic compounds. Another embodiment includes a composition which comprises a complex and a pharmaceutically acceptable excipient or carrier. Another embodiment of the instant invention relates to compositions comprising an insoluble phase suspended in a solution phase, wherein the insoluble phase is a complex comprising a protein crystal, an ionic compound and an excipient and wherein the solution phase is selected from the group consisting of: water, buffer, preservative, isotonicity agents, stabilizers and combinations thereof. Additionally, the present invention provides methods for producing these complexes and compositions.

The present invention describes complexes and compositions that have prolonged dissolution characteristics as compared with their protein crystal counterparts or with their conventionally formulated protein counterparts. The dissolution behavior is accomplished by the addition of polycationic or polyanionic compounds to a protein crystal either before or after the crystallization step. The choice of using a polycationic or polyanionic compound will depend on the pI of a protein and the pH of the crystallization environment.

In addition to complexing ionic compounds to protein crystals, it is also possible to prepare complexes and compositions of amorphous protein precipitate and spherical protein particles with polycations and polyanions.

According to this invention, the development of different crystals of proteins for sustained release compositions will rely on the amphoteric nature of proteins. For example, the number of basic and acidic residues available on a protein chain, as well as the pH environment, will determine a protein's overall net charge. Thus, inherent to every protein is an isoelectric pH value (pI) or a specific pH where the net charge of the protein is zero. The complexation of polycations or polyanions to proteins having a low pI or high pI, respectively, can result in the protein having advantageous physical properties, including favorable dissolution behavior.

For example, if human growth hormone (pI=5.2) is crystallized or precipitated in a buffer at pH 7, the protein would be negatively charged and therefore, would interact or complex with polycations. Similarly, monoclonal antibodies, such as Rituxan and Herceptin, with pIs greater than 9, would be able to complex with polyanions in neutral buffers.

The estimation of a protein net charge can be calculated once the amino acid sequence is ascertained. Publicly available programs can be accessed to accomplish this (see http://www-biol.univ-mrs.fr/d_abim/compo-p.html and http://www.infobiogen.fr/service/deambulum). Acidic proteins, those proteins having a higher content of aspartic acid (pKa 4.5) and glutamic acid (pKa 4.5), typically have pIs lower than 6 to 6.4. On the other hand, basic proteins, those proteins having a higher content of histidine (pKa 6.2), lysine (pKa 10.4) and arginine (pKa 12), typically have pIs greater than about 7.5 to 8. In contrast to both, neutral proteins, those typically having similar amounts of acid and basic amino acid residues, have pIs that are neutral (pIs are typically about 6.5 to 7.4).

Although not a comprehensive list, some examples of pI for various therapeutic proteins are as follows: recombinant human erythropoietin (pI=4); Etanercept (Enbrel) (pI=5.1); insulin (pI=5.4); granulocyte colony stimulating factor (pI=5.5-5.9); TNF-α (pI=5.6); fibrolase (pI=6.7); IL-1 β (pI=6.9); recombinant tissue plasminogen activator (pI=6.5-8.5); Orthoclone OKT3 (pI=6.7-7.2); factor VIII (pI=7-7.6); bovine somtotropin (pI=7.4); Interleukin 2 (pI=7.44); Insulin-like growth factor-1 (pI=8.4) and Aprotinin (pI=10.5).

One embodiment of this invention relates to a complex comprising a protein crystal and an ionic compound. According to an embodiment of this invention, the protein crystal and ionic compound are present in a molar ratio of protein:ionic compound of about 1:250 to about 1:20. In another embodiment, the protein crystal and ionic compound are present in a protein:ionic compound ratio of about 5:1 to about 40:1 (w/w). In another embodiment, the protein crystal and ionic compound are present in a protein:ionic compound ratio of about 10:1 to about 20:1 (w/w). In another embodiment, the protein crystal and ionic compound are present in a protein:ionic compound ratio of about 12:1 to about 15:1 (w/w). According to an alternate embodiment, that ratio is 5:1 (w/w).

In a preferred embodiment, the protein crystal is selected from the group consisting of: therapeutic proteins, fusion proteins, glycoproteins, receptors, synthetic antigens, recombinant antigens, viral surface proteins, hormones, antibodies, enzymes, Fab fragments, cyclic peptides and linear peptides.

In a more preferred embodiment, the therapeutic protein is selected from the group consisting of: glucagon-like peptide 1, antibodies, histcompatibility antigens, integrins, selecting, inhibitors, growth factors, postridical hormones, nerve growth hormones, blood clotting factors (e.g., Factor IX), adhesion molecules, bone morphogenic proteins and lectins, trophic factors, cytokines such as TGF-β, IL-2, IL-4, α-IFN, β-IFN, γ-IFN, TNF, IL-6, IL-8, lymphotoxin, IL-5, Migration inhibition factor, GMCSF, IL-7, IL-3, monocyte-macrophage colony stimulating factors, granulocyte colony stimulating factors (e.g., CSF 3), multidrug resistance proteins, other lymphokines, erythropoietin, Factor VIII, amylin, TPA, dornase-α, α-1-antitrypsin, human growth hormones, nerve growth hormones, bone morphogenic proteins, urease and toxoids.

In yet another more preferred embodiment, the therapeutic protein is selected from the group consisting of: glucagon-like peptide 1, antibodies, histcompatibility antigens, integrins, selectins, inhibitors, growth factors, postridical hormones, nerve growth hormones, blood clotting factors (e.g., Factor IX), adhesion molecules, bone morphogenic proteins and lectins, trophic factors, cytokines such as TGF-β, IL-2, IL-4, TNF, IL-6, IL-8, lymphotoxin, IL-5, Migration inhibition factor, IL-7, IL-3, monocyte-macrophage colony stimulating factors, multidrug resistance proteins, other lymphokines, Factor VIII, amylin, TPA, dornase-α, α-1-antitrypsin, human growth hormones, nerve growth hormones, bone morphogenic proteins, urease and toxoids.

In another preferred embodiment, the hormone is selected from the group consisting of: human growth hormone, glucagons, parathyroid hormone, fertility hormones, lutenizing hormone and follicle stimulating hormone.

In yet another preferred embodiment, the antibody is selected from the group consisting of: Infliximab, Entanercept (Enbrel), Rituximab, trastuzumab, Abciximab, Palivizumab, Murumonab-CD3, Gemtuzumab, Basiliximab, Daclizumab, Zevalin and Mylotarg.

In yet another preferred embodiment, the enzyme is selected from the group consisting of: rasburicase, lipase, amylase, hydrolases, oxidases, isomerases, lyases, ligases, adenylate cyclases, transferases, oxidoreductases, nitrilases, laccase, dehydrogenase, peroxidases and hydantoinase.

In a more preferred embodiment, the amylase is derived from Aspergillus oryzae. In another preferred embodiment, the lipase is derived from Burkholderia cepacia. In yet another preferred embodiment, the oxidase is selected from the group consisting of oxalate oxidase or urate oxidase (uricase).

In one embodiment, the lyase is histidase and the hydrolase is L-asparaginase II. In another embodiment, the enzyme is adenosine deaminase or ceredase.

According to one embodiment, the complexes of protein crystals and ionic polymers of this invention are not protein crystals having multilayer coatings of sequentially absorbed, oppositely charged polyelectrolytes (EP 1,190,123 B1).

The ionic compound component of the complex is selected from the group consisting of: polymers, polypeptides, oligopeptides, proteins and dendrimers. In a preferred embodiment, the oligopeptide has a molecular weight of less than about 2 kD and similarly, the polypeptide or protein has a molecular weight of greater than about 2 kD. Furthermore, the oligopeptide or polypeptide or protein component of the ionic compound of the present invention can be selected from the group consisting of polycations and polyanions.

In a more preferred embodiment, the polycation is selected from the group consisting of Protamine, polyarginine, polylysine, polyhistidine, histones, myelinbasic protein, polymyxin B sulfate, dodecyltrimethylammonium bromide, bradykinin, spermine, putrescine, octylarginine and synthetic peptides and dendrimers. In another more preferred embodiment, the polyanion is selected from the group consisting of: polyglutamate, polyaspartate, polyacrylate, polycyanoacrylates, polylactate, poly-B-hydroxybutyrate, polyvinylpyrollidone, hyaluronic acid, heparin, sulfated polysaccharides, dextran sulfates, heparin sulfates and dendrimers.

Another embodiment of the present invention relates to a composition comprising an insoluble phase suspended in a solution phase, wherein said insoluble phase is a complex comprising a protein crystal, an ionic compound and an excipient and wherein said solution phase is selected from the group consisting of: water, buffer, preservative, isotonicity agents, stabilizers and combinations thereof. Alternatively, such a composition may also be prepared without an excipient.

Additionally, a preferred embodiment of the invention also includes a composition wherein the excipient is selected from the group consisting of: detergents, pluronic polyols, polyols, glycoaminoglycans, amino acids, starch, glycerol, sugars, cellulose, povidone dextrin, polysorbates, hydroxypropyl cellulose and ascorbic acid.

Another preferred embodiment includes a composition wherein the stabilizer is selected from the group consisting of: sugars, polyols, amino acids, soluble proteins and detergents.

The present invention further provides methods of administering complexes or compositions to a mammal having a disease state requiring or ameliorated by sustained release of protein-based therapies. The method comprises the step of administering to the mammal a therapeutically effective amount of a complex comprising protein crystals and ionic compounds according to this invention. Alternatively, the method comprises the step of administering to the mammal an effective amount of a composition comprising protein crystals complexed with ionic compounds and an excipient.

In one embodiment of the invention, complexes of protein crystals and ionic compounds and compositions comprising them, with or without an excipient, are administered alone, or as part of a pharmaceutical, veterinary or prophylactic preparation. They may be administered by parenteral, oral, pulmonary, nasal, aural, anal, vaginal, dermal, ocular, intravenous, intramuscular, intraarterial, intraperitoneal, mucosal, sublingal, subcutaneous, transdermal, topical, buccal or intracranial routes.

In one embodiment of the invention, protein crystals and ionic compounds and compositions comprising them, with or without an excipient, are administered by oral route or parenteral route. In a preferred embodiment, complexes comprising protein crystals and ionic compounds and compositions comprising them, with or without an excipient, are administered by subcutaneous or intramuscular route.

In a preferred embodiment, the complexes or compositions of this invention, are administered by subcutaneous route using a needle having a gauge greater than or equal to 27. Alternatively, the complexes or compositions may be administered by needle-free injection or by transdermal means.

This invention advantageously permits sustained release of complexes or compositions of this invention into a mammal. In one embodiment, the complexes or compositions according to this invention are administered once a week. In another embodiment, the complexes or compositions according to this invention are administered every two weeks. In yet another embodiment, the complexes or compositions according to this invention are administered once every month. It will be appreciated by those of skill in the art that the specific treatment regimen will depend upon factors, such as the pharmacokinetic properties of the complex, the disease to be treated, the age and weight of the patient to be treated, general physical condition of the patient and judgment of the treating physician.

The present invention further provides methods for preparing complexes of protein crystals and ionic compounds. One such method comprises the steps of: (a) mixing a solution of a protein with a crystallization reagent mix to produce a solution; (b) adding deionized water to said solution; (c) incubating said solution for between about 2 and about 48 hours at a temperature between about 4° C. and about 40° C., until protein crystals are formed; and (d) adding an ionic compound to said solution from a complex of said protein crystals with said ionic compound.

Typically, crystallization of a protein is more likely to occur at the pH of solution near a protein's pI, wherein the protein's overall charge is zero. However, the addition of polyanions or polycations to protein crystals at their pI values may result in poor complexation at that pH. As a result, the complex of protein crystals and ionic polymer may be weak and not useful for administration in a mammal. For this reason, it is advantageous to include an additional optional step between the crystallization and complexation steps, steps (c) and (d) respectively, of the above-identified method. In that additional optional step, an additional excipient is added to the suspension of protein crystals formed in step (c) in order to reduce solubility of protein crystals at pHs different from the pH of crystallization, while at the same time changing the charge of the crystals and thus enhancing the subsequent complexation with a polyion in step (d). The resulting complex can then be formulated for parenteral administration, wherein the pH must be at a physiological pH, e.g., pH 6.5 to 7.5, or any other pH suitable for oral administration. According to an alternate embodiment, the above-described optional step includes adding an excipient to said solution to maintain crystallinity of the protein crystals but to change the pH of the protein crystals between steps (c) and (d).

The present invention also provides an alternate method for preparing complexes of protein crystals and ionic compounds. This method comprises the steps of: (a) mixing a solution of a protein with a crystallization reagent mix to produce a solution; (b) adding deionized water to said solution; (c) adding an ionic compound to said solution; and (d) incubating said solution for between about 2 and about 48 hours at a temperature between about 4° C. and about 40° C., until protein crystals are formed. A complex prepared by any method of this invention may be a co-crystal of the protein and ionic compound or may be only a physical association (i.e., electrostatic interactions) of protein crystal and ionic compound.

In another embodiment related to the above-identified methods for preparing complexes of protein crystals and ionic compounds, an excipient or a salt can be added to the solution between steps (b) and (c).

In another preferred embodiment, in the step comprising adding the ionic compound in the above-identified methods, the polycation and polyanion are added in a ratio of protein:polyanion or polycation (mg:mg) between about 1:5 to about 1:25.

If an excipient is added between steps (b) and (c) of the above-identified methods, preferred excipients include detergents, pluronic polyols, polyols, glycoaminoglycans, amino acids, starch, glycerol, sugars, cellulose, povidone dextrin, polysorbates, hydroxypropyl cellulose and ascorbic acid.

In a preferred embodiment, the protein in step (a) of the above-identified methods is present in said solution at a concentration between about 0.5 mg/ml and about 200 mg/ml.

In another preferred embodiment, the crystallization reagent mix in step (a) of the above-identified methods is selected from the group consisting of Tris-HCl, HEPES, acetate, phosphate, citrate borate, imidazole, Bis-tris, bicarbonate, carbonate, N-(2-acetamido)-iminodiacetic acid and MES. In yet another preferred embodiment, the crystallization reagent mix is present in the solution at a concentration between about 0.5 mM and about 500 mM. In another preferred embodiment, the crystalliza-tion reagent mix has a pH between about 2 and about 10.

In another preferred embodiment, the pH of the solution in step (d) of the above-identified methods is the same as said crystallization reagent mix.

In another embodiment of the above-identified methods, the solution is incubated for between about one and about two days at a temperature between about 4° C. and about 37° C.

The present invention also provides an embodiment for preparing a composition comprising a protein complex suspended in a solution phase, comprising the step of mixing said complex prepared according to the above-identified methods in a solution phase selected from the group consisting of: water, buffer, preservative, isotonicity agents, stabilizers and combinations thereof.

In a preferred embodiment, the stabilizer is selected from the group consisting of: sugars, polyols, amino acids, soluble proteins, detergents and combinations thereof.

In order that this invention may be better understood, the following examples are set forth. These examples are for the purpose of illustration only and are not to be construed as limiting the scope of the invention in any manner.

### EXAMPLES

The following materials were used in the examples set forth below.

Materials

Commercially available recombinant human growth hormone (rhGH) was from BresaGen Ltd. (Thebarton, Australia), polyethylene glycol with average molecular weight of 6000 (PEG-6000) was from Hampton Research (Laguna Niguel, Calif.) and Protamine sulfate was purchased through Fisher from ICN Biomedicals Inc. (Pittsburgh, Pa.). Ammonium phosphate, Tris-HCl, sodium citrate, dibasic sodium phosphate, calcium acetate, calcium chloride, HEPES, sodium chloride, potassium chloride and sodium azide monomethyl ether were each obtained from Fisher (Pittsburgh, Pa.). Polyarginine was obtained from Sigma (St. Louis, Mo.).

Analytical Techniques and Assays

Reverse Phase High Performance Liquid Chromatography. Reversed phase high performance liquid chromatograms (RP-HPLC) were acquired on an Agilent 1100 series HPLC (Palo Alto, Calif.) equipped with a C5, 5 cm×4.6 mm, 3 μm column (Supelco, Bellefonte, Pa.). Samples were dissolved in of dissolution buffer (50 mM HEPES pH 7.2, 140 mM NaCl, 10 mM KCl and 0.02% (v/v) NaN₃) and filtered (0.2 μm) prior to injection. Elution profiles were monitored at 214 and 280 nm using gradient method of solvents A and B. Solvent A consisted of 99.9% dH₂O/0.1% TFA. Solvent B consisted of 99.9% Acetonitrile/0.1% TFA. All chemicals were HPLC grade obtained from Fisher. Elutions were performed over 15 min. using a gradient design of 0-2 min 40-50% B, 2-12 min 50-60% B, and 12-15 60-85% B. A flow rate of 1 ml/min and a column temperature of 20° C. was maintained throughout the run. Data was analyzed using Agilent Chemstation software (Palo Alto, Calif.).

Size Exclusion Chromatography. High performance size exclusion chromatograms (SEC-HPLC) were acquired on an Agilent 1100 series HPLC (Palo Alto, Calif.) equipped with a TSK-Gel G2000SWXL column (part# 08450, Tosoh Biosep LLC, Montgomeryville, Pa.) (7.8 mm×30 cm, 5 μm) and an Agilent 1100 series MWD (UV). Samples were dissolved in 0.2 ml of dissolution buffer and 0.2 μm filtered prior to injection into Agilent 1100 series temperature controlled Autosampler. Elution profiles were monitored at 214 and 280 nm, with a mobile phase of 50 mM Tris-HCl, 150 mM Nacl, 0.05% NaN₃, pH 7.5. Column temperature was maintained at 25° C., solvents were degassed using an Agilent 1100 series degasser.

UV-VIS absorption and Optical Microscopy. Uv-VIS spectrophotographs were obtained on a Beckman DU 7400 spectrophotometer, Beckman Coulter Inc., Fullerton, Calif. Optical micrographs were obtained by bright field imaging using an Olympus BX-51 microscope and captured by a Sony DXC-970MD 3CCD color digital video camera using Image-Pro software, Media Cybernetics L. P., Silver Springs, Md., under the magnifications of 40× to 400×.

### Example 1

Crystallization of hGH with ammonium phosphate. Commercially available hGH (50 mg) was first dissolved in 15 ml Tris-HCl (10 mM, pH 8.0) and dialyzed against 2×4000 ml Tris-HCl (10 mM, pH 8.0) using a Pierce Dialyzer cartridge having a molecular weight cutoff (MWCO) of 10,000. Protein concentration was adjusted by centrifugation using a Millipore concentrator (MWCO 10,000) at 4000 rpm for 20-30 minutes. The concentration of hGH was found in a range of 30-45 mg/ml, as measured by absorbance at 280 nm/0.813 (1 mg/ml hGH A₂₈₀=0.813 absorbance units) Deionized water was added to the solution to yield a final protein concentration of 10-20 mg/ml. Crystals of hGH were grown by adding ammonium phosphate (NH₄H₂PO₄) (2.5 M) to the solution, so that a final concentration of 860 mM NH₄H₂PO₄ was obtained. The solution was then incubated for 16 hours at 25° C. Needle-like crystals were obtained and imaged by optical microscopy. The crystals obtained were found to be approximately 8 to 15 μm in length, with a crystallization yield of greater than 90%.

### Example 2

Crystallization of hGH with sodium citrate. Commercially available hGH was purified and/or dialyzed and concentrated as described in Example 1. Deionized water was added to the concentrated solution of hGH to yield a final protein concentration of 17.5 mg/ml. Crystals of hGH were grown by adding sodium citrate (Na-Citrate) (1.5 M) to the solution so that a final concentration of 390 mM Na-Citrate was obtained. The solution was then incubated for 16 hours at 25° C. Needle-like crystals were obtained and imaged by optical microscopy. The crystals obtained were found to be less than 8 μm in length with a crystallization yield of greater than 85%.

### Example 3

Crystallization of hGH with sodium phosphate. Commercially available hGH was purified and/or dialyzed and concentrated as described in Example 1. Deionized water was added to the concentrated hGH solution to yield a final protein concentration of 12.5-17.5 mg/ml. Tris-HCl (1M, pH 8.6) was added to a final concentration of 100 mM. Crystals of hGH were grown by adding dibasic sodium phosphate (Na₂HPO₄) (1 M) to the solution so that a final concentration of 600 mM Na₂HPO₄ was obtained. The solution was then incubated for 16 hours at 25° C. Needle-like crystals were obtained and imaged by optical microscopy. The crystals obtained were found to be between 5 and 25 μm in length with a crystallization yield of greater than 75%.

### Example 4

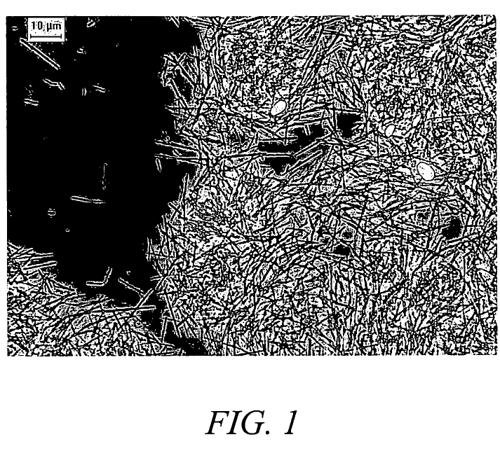

Crystallization of hGH with calcium acetate and Protamine sulfate. Commercially available hGH was purified and/or dialyzed and concentrated as described in Example 1. Deionized water was added to the concentrated hGH solution to yield a final protein concentration of 15 mg/ml. Tris-HCl (1M, pH 8.6) was added to a final concentration of 100 mM. To this solution, Protamine sulfate was added to final concentration of 2 mg/ml. Crystals of hGH were grown by adding calcium acetate (Ca-Acetate) (1 M) to the solution so that a final concentration of 85 mM Ca-Acetate was obtained. The solution was then incubated for 8 hours at 37° C. Needle-like crystals were obtained and imaged by optical microscopy. The crystals obtained were found to be less than 20 μm in length with a crystallization yield of greater than 70%. See FIG. 1.

### Example 5

Solubility profile of hGH crystals prepared by salt induced crystallization. After the incubation of the crystallization solutions in Examples 1-4, the crystals were pelleted and the remaining supernatant removed. The crystal pellets were resuspended in 0.200 ml of dissolution buffer (50 mM HEPES (pH 7.2), 140 mM NaCl, 10 mM KCl and 0.02% (v/v) NaN₃) by either pipetting or vortexing before being equilibrated for approximately 15 minutes at 37° C. Protein concentration after pellet resuspension was approximately 2 mg/ml. The samples were then centrifuged at 10,000×g for 2 minutes and the supernatant was completely removed for determination of protein concentration measured at 280 nm by RP-HPLC, SEC-HPLC or UV-VIS. The crystalline pellets were further resuspended in 0.200 ml of dissolution buffer and the process repeated until no detectable protein was measured in the supernatant. This process is referred to as sequential dissolution.

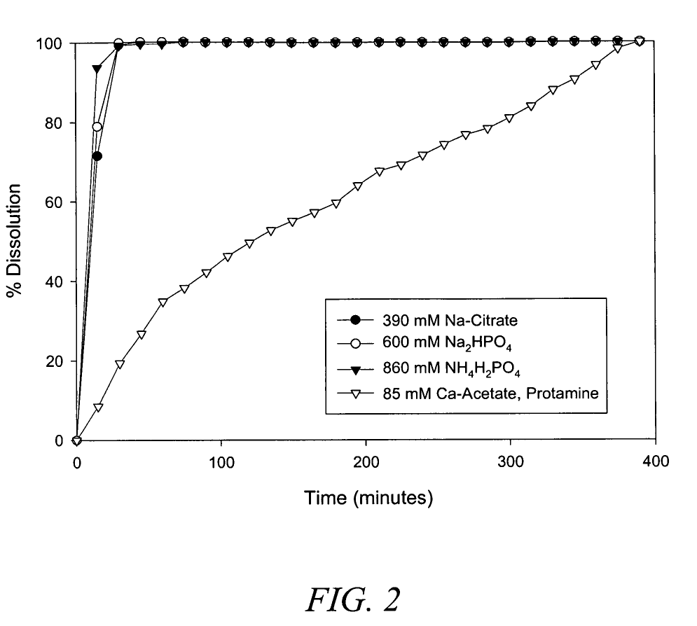

FIG. 2 shows the solubility behavior of various hGH crystals prepared with monovalent (Na or NH₄) or divalent (Ca) salts in Examples 1-4 above as a function of time in minutes. hGH dissolution was measured as a cumulative percentage and derived from AUC values or UV-VIS mg/ml measurements. The results demonstrate that divalent calcium crystals of hGH dissolve at a significantly slower rate than monovalent sodium or ammonium crystals of hGH. The data illustrates that hGH crystals prepared by the addition of 390 mM Na-Citrate are completely dissolved after 60 minutes. In addition, hGH crystals prepared by the addition of 600 mM Na₂HPO₄ or 860 mM NH₄H₂PO₄ are completely dissolved after 60 or 75 minutes, respectively. On the other hand, hGH crystals prepared by the addition of 85 mM Ca-Acetate and Protamine sulfate dissolved completely after 390 minutes (refer to Table 1 below).

TABLE 1
Sequential dissolution test measured at 280 nm
for salts of hGH in dissolution buffer

| | | | | 85 mM Ca- |
|---|---|---|---|---|
| | 390 mM Na- | 600 mM | 860 mM | Acetate + |
| Time | Citrate | Na₂HPO₄ | NH₄H₂PO₄ | Protamine |
| (minutes) | (Ex. 2) | (Ex. 3) | (Ex. 1) | (Ex. 4) |
| 0 | 0.00 | 0.00 | 0.00 | 0.00 |
| 15 | 71.59 | 78.99 | 93.77 | 8.53 |
| 30 | 99.36 | 99.85 | 99.18 | 19.39 |
| 45 | 99.99 | 99.99 | 99.50 | 26.81 |
| 60 | 100.00 | 100.00 | 99.50 | 34.92 |
| 75 | 100.00 | 100.00 | 100.00 | 38.31 |
| 90 | 100.00 | 100.00 | 100.00 | 42.22 |
| 105 | 100.00 | 100.00 | 100.00 | 46.26 |
| 120 | 100.00 | 100.00 | 100.00 | 49.62 |
| 135 | 100.00 | 100.00 | 100.00 | 52.73 |
| 150 | 100.00 | 100.00 | 100.00 | 55.08 |
| 165 | 100.00 | 100.00 | 100.00 | 57.20 |
| 180 | 100.00 | 100.00 | 100.00 | 59.65 |
| 195 | 100.00 | 100.00 | 100.00 | 63.95 |
| 210 | 100.00 | 100.00 | 100.00 | 67.57 |
| 225 | 100.00 | 100.00 | 100.00 | 69.17 |
| 240 | 100.00 | 100.00 | 100.00 | 71.63 |
| 255 | 100.00 | 100.00 | 100.00 | 74.35 |
| 270 | 100.00 | 100.00 | 100.00 | 76.85 |
| 285 | 100.00 | 100.00 | 100.00 | 78.39 |
| 300 | 100.00 | 100.00 | 100.00 | 81.06 |
| 315 | 100.00 | 100.00 | 100.00 | 83.97 |
| 330 | 100.00 | 100.00 | 100.00 | 87.97 |
| 345 | 100.00 | 100.00 | 100.00 | 90.57 |
| 360 | 100.00 | 100.00 | 100.00 | 94.20 |
| 375 | 100.00 | 100.00 | 100.00 | 98.28 |
| 390 | 100.00 | 100.00 | 100.00 | 100.00 |

### Example 6

Crystallization of hGH with calcium acetate and 2% PEG-6000. Commercially available hGH was purified and/or dialyzed and concentrated as described in Example 1. Deionized water was added to the concentrated hGH solution to yield a final protein concentration of 15 mg/ml. Tris-HCl (1M, pH 8.6) was added to a final concentration of 100 mM. To this solution, 2% (v/v) PEG-6000 was added. Crystals of hGH were grown by adding Ca-Acetate (1 M) to the solution so that a final concentration of 85 mM Ca-Acetate was obtained. The solution was then incubated for 16 hours at 25° C. Needle-like crystals were obtained and imaged by optical microscopy. The crystals obtained were found to be between about 25 and about 75 μm in length with a crystallization yield of greater than 85%.

### Example 7

Crystallization of hGH with sodium acetate and 6% PEG-6000. Commercially available hGH was purified and/or dialyzed and concentrated as described in Example 1. Deionized water was added to the concentrated hGH solution to yield a final protein concentration of 15 mg/ml. Tris-HCl (1M, pH 8.6) was added to a final concentration of 100 mM. To this solution, 6% (v/v) PEG-6000 was added. Crystals of hGH were grown by adding sodium acetate (Na-Acetate) (2 M) to the solution so that a final concentration of 500 mM Na-Acetate was obtained. The solution was then incubated for 16 hours at 25° C. Needle-like crystals were obtained and imaged by optical microscopy. The crystals obtained were found to be between about 25 and about 75 μm in length with a crystallization yield of greater than 85%.

### Example 8

Crystallization of hGH with calcium chloride and 6% PEG-6000. Commercially available hGH was purified and/or dialyzed and concentrated as described in Example 1. Deionized water was added to the concentrated hGH solution to yield a final protein concentration of 15 mg/ml. Tris-HCl (1 M, pH 8.6) was added to a final concentration of 100 mM. To this solution, 6% (v/v) PEG-6000 was added. Crystals of hGH were grown by adding CaCl₂ (1 M) to the solution, so that a final concentration of 85 mM CaCl₂ was obtained. The solution was then incubated for 16 hours at 25° C. Needle-like crystals were obtained and imaged by optical microscopy. The crystals obtained were found to be between greater than 100 μm in length with a crystallization yield of greater than 90%.

### Example 9

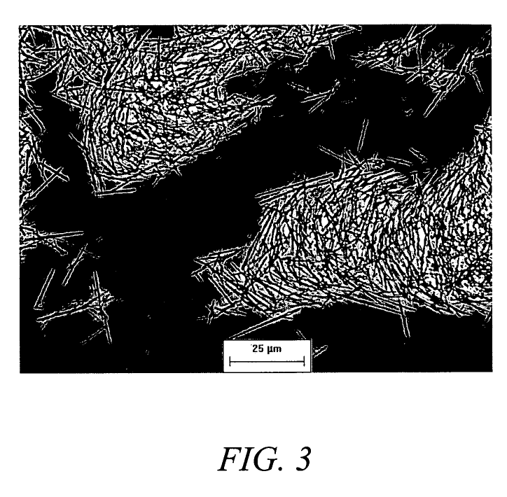

Crystallization of hGH with calcium acetate, 6% PEG-6000 and Protamine sulfate. Commercially available hGH was purified and/or dialyzed and concentrated as described in Example 1. Deionized water was added to the concentrated hGH solution to yield a final protein concentration of 15 mg/ml. Tris-HCl (1M, pH 8.6) was added to a final concentration of 100 mM. To this solution, Protamine sulfate (1 mg/ml) and 6% PEG-6000 (v/v) was added. Crystals of hGH were grown by adding Ca-Acetate (1 M) to the solution so that a final concentration of 85 mM Ca-Acetate was obtained. The solution was then incubated for 16 hours at 37° C. Needle-like crystals were obtained and imaged by optical microscopy. The crystals obtained were found to be less than 25 μm in length with a crystallization yield of greater than 70%. See FIG. 3.

### Example 10

Crystallization of hGH with calcium acetate and 6% PEG-MME-5000. Commercially available hGH was purified and/or dialyzed and concentrated as described in Example 1. Deionized water was added to the concentrated hGH solution to yield a final protein concentration of 15 mg/ml. Tris-HCl (1 M, pH 8.6) was added to a final concentration of 100 mM. To this solution, 6% (v/v) polyethylene glycol mono methyl ether-5000 (PEG-MME-5000) was added. Crystals of hGH were grown by adding Ca-Acetate (1 M) to the solution so that a final concentration of 125 mM Ca-Acetate was obtained. The solution was then incubated for 16 hours at 25° C. Needle-like crystals were obtained and imaged by optical microscopy. The crystals obtained were found to be less than 50 μm in length with a crystallization yield of greater than 90%.

### Example 11

Solubility profile of hGH crystals prepared with polyethylene glycol. After the incubation of the crystallization solutions prepared in Examples 6-10, the crystals were pelleted and the remaining supernatant removed. The crystal pellets were resuspended in 0.2 ml of dissolution buffer (see Example 5) by either pipetting or vortexing before being equilibrated for approximately 15 minutes at 37° C. The samples were then centrifuged at 10,000×g for 2 minutes and the supernatant was removed for determination of protein concentration measured at 280 nm by RP-HPLC, SEC-HPLC or UV-VIS. The crystalline pellets were further resuspended in dissolution buffer and the process repeated until no detectable protein was measured in the supernatant.

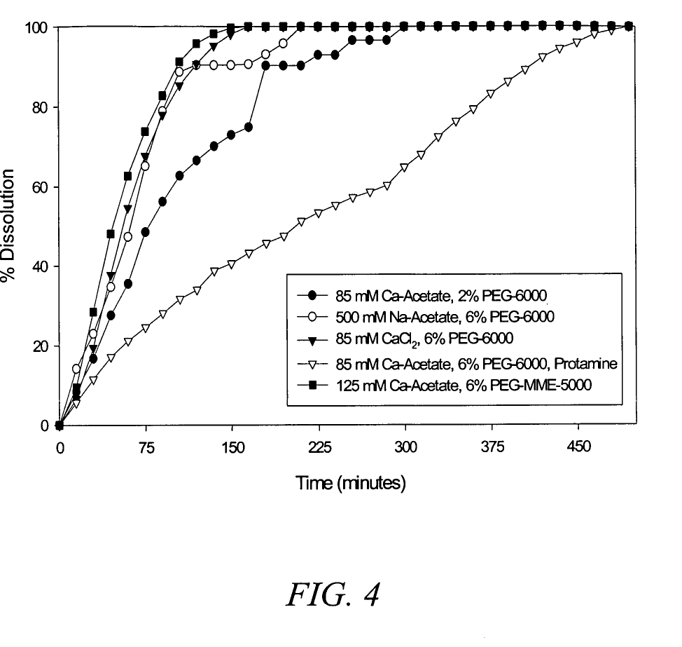

FIG. 4 and Table 2 illustrate the solubility behavior of hGH crystals prepared with 2% PEG-6000/85 mM Ca-Acetate, 6% PEG-6000/500 mM Na-Acetate, 6% PEG-6000/85 mM CaCl₂, 6% PEG-6000/85 mM Ca-Acetate/Protamine and 6% PEG-MME-5000/125 mM Ca-Acetate as a function of time in minutes. hGH dissolution was measured as a cumulative percentage and derived from AUC values or UV-VIS mg/ml measurements. The results demonstrate that the hGH crystals prepared by the addition of 6% PEG-6000/85 mM Ca-Acetate/Protamine are the slowest to dissolve, with complete dissolution occurring after 495 minutes. The other crystals dissolved at 300 minutes for 2% PEG-6000/85 mM Ca-Acetate crystals or less for the other hGH crystals.

TABLE 2
Sequential dissolution test measured at 280 nm for
PEG and salts of hGH in dissolution buffer

| | | | | 6% PEG- | 6% PEG- |
|---|---|---|---|---|---|
| | 2% PEG- | 6% PEG- | 6% PEG- | 6000/85 mM | MME- |
| | 6000/85 mM | 6000/500 mM | 6000/85 mM | Ca-Acetate/ | 5000/125 mM |
| | Ca- | Na- | Ca- | Protamine | Ca- |
| Time | Acetate | Acetate | Chloride | sulfate | Acetate |
| (minutes) | (Ex. 6) | (Ex. 7) | (Ex. 8) | (Ex. 9) | (Ex. 10) |
| 0 | 0.00 | 0.00 | 0.00 | 0.00 | 0.00 |
| 15 | 8.41 | 14.23 | 6.63 | 5.66 | 9.50 |
| 30 | 16.80 | 23.03 | 19.50 | 11.58 | 28.46 |
| 45 | 27.64 | 34.74 | 37.74 | 17.22 | 48.04 |
| 60 | 35.57 | 47.34 | 54.60 | 21.25 | 62.61 |
| 75 | 48.57 | 65.16 | 67.67 | 24.63 | 73.76 |
| 90 | 56.18 | 78.86 | 77.90 | 28.15 | 82.70 |
| 105 | 62.70 | 88.66 | 85.26 | 31.77 | 91.15 |
| 120 | 66.49 | 90.36 | 90.59 | 34.05 | 95.70 |
| 135 | 70.07 | 90.36 | 95.18 | 38.83 | 98.18 |
| 150 | 72.87 | 90.36 | 98.04 | 40.60 | 99.60 |
| 165 | 74.82 | 90.58 | 100.00 | 43.28 | 100.00 |
| 180 | 90.23 | 93.06 | 100.00 | 45.69 | 100.00 |
| 195 | 90.23 | 95.80 | 100.00 | 47.52 | 100.00 |
| 210 | 90.23 | 100.00 | 100.00 | 51.27 | 100.00 |
| 225 | 92.90 | 100.00 | 100.00 | 53.38 | 100.00 |
| 240 | 92.90 | 100.00 | 100.00 | 55.31 | 100.00 |
| 255 | 96.61 | 100.00 | 100.00 | 57.24 | 100.00 |
| 270 | 96.61 | 100.00 | 100.00 | 58.61 | 100.00 |
| 285 | 96.61 | 100.00 | 100.00 | 60.28 | 100.00 |
| 300 | 100.00 | 100.00 | 100.00 | 64.90 | 100.00 |
| 315 | 100.00 | 100.00 | 100.00 | 68.04 | 100.00 |
| 330 | 100.00 | 100.00 | 100.00 | 72.46 | 100.00 |
| 345 | 100.00 | 100.00 | 100.00 | 76.26 | 100.00 |
| 360 | 100.00 | 100.00 | 100.00 | 79.36 | 100.00 |
| 375 | 100.00 | 100.00 | 100.00 | 83.20 | 100.00 |
| 390 | 100.00 | 100.00 | 100.00 | 86.17 | 100.00 |
| 405 | 100.00 | 100.00 | 100.00 | 89.15 | 100.00 |
| 420 | 100.00 | 100.00 | 100.00 | 92.25 | 100.00 |
| 435 | 100.00 | 100.00 | 100.00 | 94.40 | 100.00 |
| 450 | 100.00 | 100.00 | 100.00 | 95.96 | 100.00 |
| 465 | 100.00 | 100.00 | 100.00 | 98.07 | 100.00 |
| 480 | 100.00 | 100.00 | 100.00 | 99.07 | 100.00 |
| 495 | 100.00 | 100.00 | 100.00 | 100.00 | 100.00 |

### Example 12

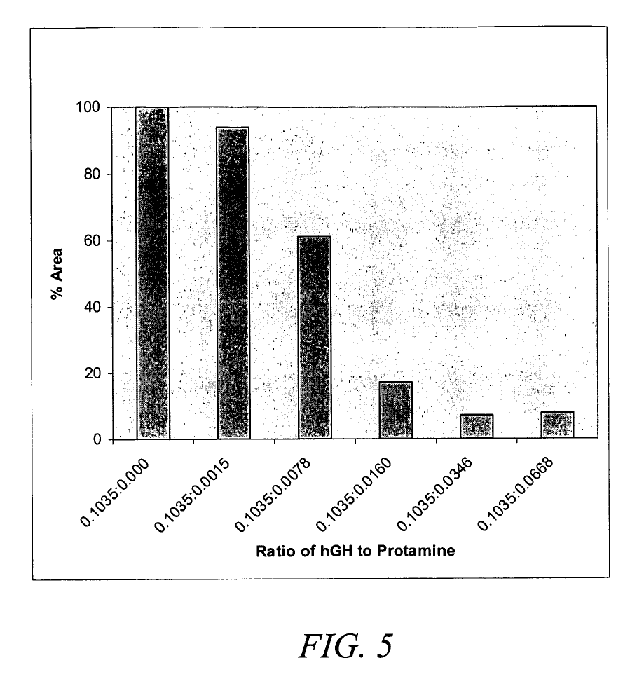

Effect of Protamine sulfate on dissolution characteristics of hGH crystals. FIG. 5 illustrates the amount of hGH crystals (85 mM calcium acetate, 2% (v/v) PEG-6000 and 100 mM Tris-HCl (pH 8.6)) dissolved after 1 hour incubation in dissolution buffer at 37° C. after adding an amount of Protamine sulfate to the pre-existing calcium hGH crystal solution. The ratios of hGH to Protamine (mg:mg) ratios are indicated in FIG. 5. The graph illustrates that Protamine significantly affects dissolution of hGH crystals.

### Example 13

Crystallization and complexation of glucose oxidase. Glucose oxidase (pI 4.6) can bind to both polycations and polyanions. Glucose oxidase (Sigma) was diafiltered in water and concentrated to 15 mg/ml. The enzyme (10 ml) was then mixed (1:1) with the crystallizing reagent containing 18% PEG-6000, 32% isopropanol in 0.2 M sodium acetate buffer at pH 5.0. After mixing, the solution was transferred to 4° C. and crystallization was allowed to proceed for 24 hrs with stirring at 100 rpm.

Glucose oxidase crystals are collected by centrifugation at 1000 rpm for 10 minutes. These crystals are then resuspended in 10% PEG-6000 and 16% isopropanol such that concentration of protein in the suspension is 15 mg/ml. The crystals are then removed into 4×1 ml aliquots and 100 μl. A 1 M buffer stock solution of glycine (pH 2.5), acetate (pH 4.6), MES (pH 6.5) or HEPES (pH 7.5) is added to one of the four aliquots. To ensure no dissolution of the crystals, concentration of protein in the supernatant is measured at A₂₈₀ nm. Any loss of crystallinity of the protein is also measured by microscopy. If necessary, the concentration of PEG or isopropanol is increased to maintain crystallinity.

To the crystal suspensions at pH 2.5, 4.6, 6.5 and 7.5, polyanions, such as polyaspartate or polyglutamate are added. For each pH, the following ratios of protein:polyanion (w/w) are tested: 0.1:1, 1:1, 5:1 and 10:1. The protein:polyanion solutions are then equilibrated overnight at 4° C. The samples are then centrifuged at 100 rpm for 10 minutes before the supernatant is removed. The resulting pellets are resuspended in 10 ml of dissolution buffer (10 mM Tris, pH 7.5) at 37° C. Control samples are prepared by taking 1 ml of protein crystals, without polyanion, removing the supernatant, and resuspending the pellet in 10 ml of dissolution buffer (10 mM Tris, pH 7.5) at 37° C. Both complexed and uncomplexed proteins are resuspended in fresh dissolution buffer every 4 hours. Protein concentration for complexed and uncomplexed protein is read over time until complete dissolution is achieved.

### Example 14

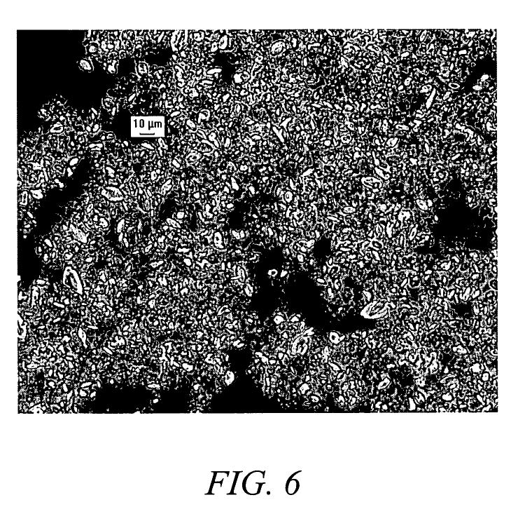

Crystallization and complexation of Rasburicase with polyarginine. Rasburicase (Biozyme) was crystallized at pH 8.5 with 5% ethanol and 15% PEG-6000 at 10 mg/ml final protein concentration. Crystals are then centrifuged at 2000 rpm for 5 minutes to remove soluble protein and then are resupended in fresh mother liquor. The crystals obtained were imaged by optical microscopy (see FIG. 6).

To 1 ml of a 5 mg/ml suspension of crystals, polyarginine is added at a protein:polymer ratio (w/w) of 1:0, 0.05:1, 1:1, 5:1 and allowed to equilibrate for 6 hrs. Subsequently, samples are centrifuged to remove supernatant, dissolved in 10 mM Tris buffer containing 150 mM NaCl at 37° C. Complete dissolution time is then measured by UV-VIS or by RP HPLC. The saturation point of protein:polymer ratio beyond which dissolution is not effected can also be measured.

To 1 ml of a 5 mg/ml suspension of crystals, polyarginine was added at a protein:polymer ratio (w/w) of 4:1 and allowed to equilibrate for 6 hrs. Subsequently, the sample was centrifuged to remove supernatant and dissolved in 10 mM Tris buffer (pH 8.5) containing 150 mM NaCl at 37° C. Complete dissolution time was then measured by UV-VIS or by RP HPLC.

### Example 15

Crystallization and complexation of Rasburicase with polylysine. Rasburicase (Biozyme) was crystallized at pH 8.5 with 5% ethanol and 15% PEG-6000 at 10 mg/ml final protein concentration. Crystals are then centrifuged at 2000 rpm for 5 minutes to remove soluble protein and then are resupended in fresh mother liquor.

To 1 ml of a 5 mg/ml suspension of crystals, polylysine is added at a protein:polymer ratio (w/w) of 1:0, 0.05:1, 1:1, 5:1 and allowed to equilibrate for 6 hrs. Subsequently, samples are centrifuged to remove supernatant, dissolved in 10 mM Tris buffer containing 150 mM NaCl at 37° C. Complete dissolution time is then measured by UV-VIS or by RP HPLC. The saturation point of protein:polymer ratio beyond which dissolution is not effected can also be measured.

### Example 16

Crystallization and complexation of Rituximab with polyaspartate and polyglutamate. Rituximab (10 mg/ml) was crystallized by mixing 1 ml of the antibody with 1 ml of solution containing 0.2 M calcium acetate, 0.1 M imidazole (pH 8.0), 10% PEG-8000. Mixture was tumbled in a hematology/chemistry mixer (Model 346, Fisher Scientific) at room temperature at 225 rpm. After 24 hrs at room temperature, Rituximab crystals having needle-like clusters were formed.

To 0.5 ml of a 5 mg/ml suspension of crystals, polyglutamate is added at a protein:polymer ratio (w/w) of 1:0, 0.05:1, 1:1, 5:1 and allowed to equilibrate for 6 hrs. Subsequently, samples are centrifuged and supernatant removed. The crystal complex is then dissolved in 10 mM Tris buffer containing 150 mM NaCl at 37° C. Complete dissolution time is then measured by UV-VIS or by RP HPLC. The saturation point of protein:polymer ratio beyond which dissolution is not effected can also be measured. Similar preparations using other polyanions may be made.

### Example 17

Crystallization and complexation of Trastuzumab with histindine, trehalose and polysorbate. 10 ml of Trastuzumab (22 mg/ml) in a buffering solution (0.495 mg/ml L-histidine HCl, 0.32 mg/ml L-Histidine, 20 mg/ml trehalose, 0.09 mg/ml polysorbate 20) was mixed with 10 ml of crystallization buffer (25% PEG-400, 10% propylene glycol, 0.1 M Tris (pH 8.5) and 5% PEG-8000) and incubated at room temperature overnight. The mixture was tumbled in a hematology mixer and further supplemented with 1 ml of propylene glycol. Trastuzumab crystals were obtained the following day.

To 0.5 ml of a 5 mg/ml suspension of crystals, polyglutamate is added at a protein:polymer ratio (w/w) of 1:0, 0.05:1, 1:1, 5:1 and allowed to equilibrate for 6 hrs. Subsequently, samples are centrifuged and supernatant removed. The crystal complex is then dissolved in 10 mM Tris buffer containing 150 mM NaCl at 37° C. Complete dissolution time is then measured by UV-VIS or by RP HPLC. The saturation point of protein:polymer ratio beyond which dissolution is not effected can also be measured.

### Example 18

Crystallization and complexation of Rasburicase with ionic polymers. Rasburicase (Biozyme) was crystallized at pH 8.5 with 5% ethanol and 15% PEG-6000 at 10 mg/ml final protein concentration. Crystals were then centrifuged at 2000 rpm for 5 minutes to remove soluble protein and then were resupended in fresh mother liquor.

Uricase (from Rasburicase above) was crystallized in a 1 ml batch containing 32 mg/ml purified uricase, 10% PEG-6000, and 25 mM glycine (pH 9.0). After an incubation period of 48 hours at 4° C., the crystals were washed with a modified mother liquor containing 15% PEG-6000, 10% ethanol, and 25 mM glycine (pH 9.0). The crystal absorbance was measured at 280 nm and the concentration of the crystal was measured as 38.8 mg/ml. Crystals were then divided into 5 aliquots of 20 μl each, each aliquot having a total weight of 0.78 mg of Rasburicase crystals per tube. To a control tube (bare Rasburicase crystals), 10 μl of modified mother liquor was added. To each of the remaining tubes, 0.2 mg (10 μl of 20 mg/ml solution) of one of the following complexation components was added: polyarginine, polyornithine, polylysine, or protamine. Each tube was then incubated overnight at 4° C.

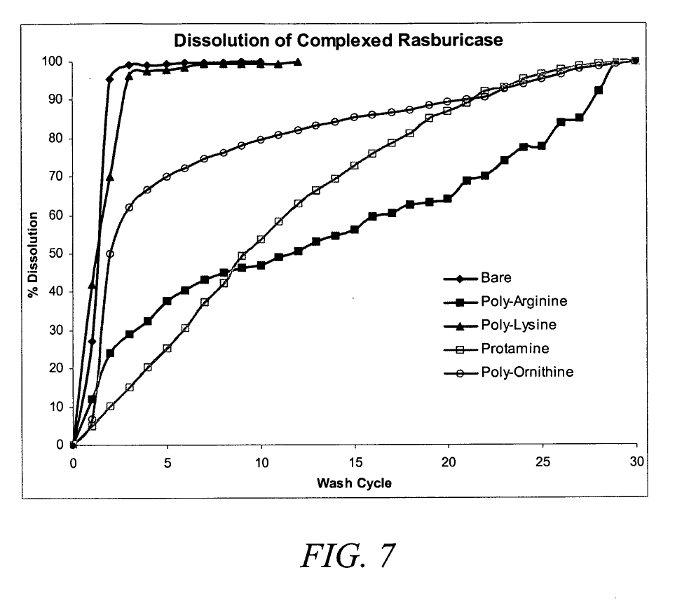

In order to study the dissolution behavior of Rasburicase complexed with various ionic polymers, the following study was designed. The crystals in each of the above tubes were washed with uricase dissolution buffer (150 mM NaCl, 10 mM Tris (pH 8.5)) in the following order: 1) 50 μl of dissolution buffer was added to each tube, 2) the resuspended crystals were incubated at room temperature for 2 minutes, 3) the tubes were then centrifuged for 10 minutes at 7000 rpm, 4) the supernatant was removed and saved for analysis and 5) steps 1-4 were repeated until complete dissolution of the crystals was achieved. The supernatant from each wash cycle was analyzed by SEC-HPLC (protein peak for 130 kD eluted at approximately 6.7 minutes) and percent cumulative dissolution for each group was reported in Table 3 and illustrated in FIG. 7.

TABLE 3
Percent Cumulative Dissolution of Rasburicase
Complexed with Ionic Polymers

| | | Rasburicase | Rasburicase | Rasburicase | Rasburicase |
|---|---|---|---|---|---|
| | | crystal | crystal | crystal | crystal |
| | | complexed | complexed | complexed | complexed |
| | Bare | with | with | with | with |
| Wash Cycle | Rasburicase crystal | polyarginine | polylysine | protamine | polyornithine |
| 0 | 0 | 0 | 0 | 0 | 0 |
| 1 | 27.0 | 11.9 | 42.0 | 5.1 | 6.6 |
| 2 | 95.5 | 23.9 | 70.0 | 10.1 | 49.9 |
| 3 | 99.1 | 29.0 | 96.4 | 15.2 | 61.9 |
| 4 | 99.2 | 32.3 | 97.5 | 20.3 | 66.6 |
| 5 | 99.5 | 37.7 | 97.9 | 25.4 | 70.0 |
| 6 | 99.6 | 40.5 | 98.4 | 30.7 | 72.1 |
| 7 | 99.8 | 43.3 | 99.4 | 37.3 | 74.6 |
| 8 | 100.0 | 45.0 | 99.4 | 42.1 | 76.3 |
| 9 | 100.0 | 46.3 | 99.4 | 49.4 | 78.2 |
| 10 | | 47.0 | 99.4 | 53.7 | 79.7 |
| 11 | | 49.2 | 99.4 | 58.3 | 80.9 |
| 12 | | 50.6 | 100.0 | 62.8 | 82.0 |
| 13 | | 53.2 | | 66.4 | 83.3 |
| 14 | | 54.6 | | 69.3 | 84.3 |
| 15 | | 56.3 | | 73.0 | 85.4 |
| 16 | | 59.5 | | 76.0 | 86.2 |
| 17 | | 60.5 | | 78.9 | 86.8 |
| 18 | | 62.7 | | 81.3 | 87.3 |
| 19 | | 63.3 | | 85.3 | 88.7 |
| 20 | | 64.3 | | 87.0 | 89.4 |
| 21 | | 68.9 | | 89.2 | 90.1 |
| 22 | | 70.1 | | 92.1 | 90.7 |
| 23 | | 74.0 | | 93.3 | 92.8 |
| 24 | | 77.4 | | 95.4 | 94.0 |
| 25 | | 77.6 | | 96.5 | 95.5 |
| 26 | | 84.0 | | 97.7 | 96.6 |
| 27 | | 85.3 | | 98.8 | 98.2 |
| 28 | | 92.1 | | 99.4 | 98.9 |
| 29 | | 99.5 | | 99.7 | 99.4 |
| 30 | | 100.0 | | 100.0 | 100.0 |

Each sample required a different number of wash cycles to completely dissolve the bare or complexed Rasburicase crystals. The untreated control or bare crystals dissolved in ten washes. The polyarginine, polyornithine, and protamine-complexed crystals of Rasburicase required 30 washes for complete dissolution of the complexed crystals. In contrast, the polylysine-complexed crystals of Rasburicase required 15 washes for complete dissolution of the complexed crystals. In addition, the polyarginine-complexed Rasburicase showed the best dissolution profile, with the crystals very gradually releasing over a long time. Polyornithine-complexed Rasburicase, while taking approximately the same number of washes to dissolve, did so with a sudden burst followed by slow dissolution. Polylysine-complexed Rasburicase displayed very little improvement over the control bare crystals, which dissolved almost immediately.

### Example 19

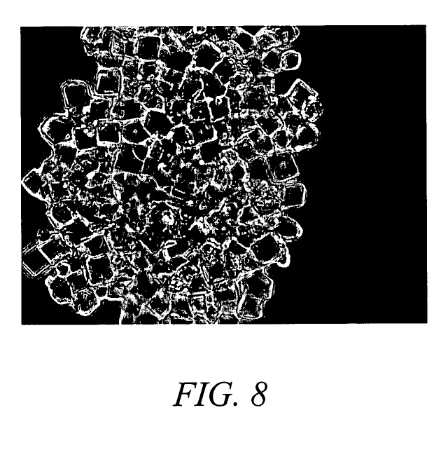

Crystallization and complexation of oxalate oxidase. Oxalate oxidase, which was minimally expressed in yeast, was concentrated to 12 mg/ml. One 1 ml portion of the enzyme was then mixed with two portions of a crystallizing reagent containing 40% PEG-600 in 100 mM phosphate citrate buffer at pH 4.2. After mixing, crystals appeared after 1 hr. The crystals obtained were imaged by optical microscopy (see FIG. 8).

After 4 hours, oxalate oxidase crystals were washed with 25% PEG-6000 and 100 mM Tris buffer (pH 8.5) and then resuspended in 25% PEG-6000 and 100 mM Tris Buffer (pH 8.5), to yield a 20 mg/ml crystal suspension. A 20 μl aliquot of crystal suspension was removed and 10 μl of 20 mg/ml polyarginine (MW=7.5 kD) added to it. The mixture was incubated for 17 hours. After that, the complex was microcentrifuged for 5 minutes and the supernatant removed. The resulting pellets were resuspended in 200 μl of dissolution buffer (50 mM HEPES, 140 mM NaCl, 10 mM KCl, pH 7.2).

### Example 20

Crystallization and complexation of Burkholderia cepacia lipase. Burkholderia cepacia lipase was diafiltered and concentrated to 44 mg/ml. One 1 ml portion of the enzyme in 100 mM sodium acetate (pH 5.5) was then mixed with one portion of a crystallizing reagent containing 50% tert-butanol. After mixing, crystals appeared within 1 hr.

After 3 hrs lipase crystals were washed with 35% Tert-butanol and 100 mM Tris buffer (pH 8.5) and then resuspended in 35% tert-butanol and 100 mM Tris buffer (pH 8.5), to yield a 22 mg/ml crystal suspension. A 20 μl aliquot of crystal suspension was removed and 10 μl of 20 mg/ml polyarginine (MW=7.5 kD) added to it. The mixture was incubated for 17 hours. After that, the complex was centrifuged for 5 minutes and the supernatant removed. The resulting pellets were resuspended in 200 μl of dissolution buffer (50 mM HEPES, 140 mM NaCl, 10 mM KCl, pH 7.2).

### Example 21

Crystallization and complexation of amylase. Amylase (Aspergillus Oryzae) was concentrated to 80 mg/ml. One 1 ml portion of the enzyme was then mixed with one portion of a crystallizing reagent containing 42% PEG-8000, in 300 mM calcium acetate at pH 6.0. After 2 hrs crystallization was complete and the crystals were washed in 25% PEG-8000 in 100 mM Tris buffer (pH 8.5) and suspended in 25% PEG-8000 in 100 mM Tris buffer (pH 8.5) to yield a 40 mg/ml crystal suspension.

After 2 hrs, a 10 μl 40 mg/ml aliquot of crystal suspension was removed and 10 μl of 40 mg/ml polyarginine (MW=9.5 kD) added to it. The mixture was incubated for 17 hours. After that, the complex was centrifuged for 5 minutes and the supernatant removed. The resulting pellets were resuspended in 200 μl of dissolution buffer (50 mM HEPES, 140 mM NaCl, 10 mM KCl, pH 7.2).

### Example 22

Crystallization and complexation of Trastuzumab. Trastuzumab (antibody CHO cell-derived) is reconstituted in water 22 mg/ml. One 1 ml portion of the antibody is then mixed with two portion of a crystallizing reagent containing 50% PEG-400, 10% PEG-8000, 20% propylene glycol, 0.2% Tween-80, 0.1M Tris pH 8.6. The mixture is incubated overnight at room temperature. Trastuzumab crystals are obtained the following day. After crystallization is complete, the crystals are washed with crystallization buffer (30% PEG-400, 7% PEG-8000, 30% propylene glycol, 0.1% Tween-80, 0.1 M MES pH 5.5). A 200 μl aliquot of crystals are removed and washed with buffer containing (MES buffer (pH 5.5).

To 20 μl of a 20 mg/ml suspension of crystals, polyglutamic acid (10 μl, 20 mg/ml, MW 90 kD] or aspartic acid (amount 10 μl, 20 mg/ml, MW 90 kD) is added. The mixture is incubated at room temperature for 17 hours. After that, the complex is microcentrifuged for 5 minutes and the supernatant removed. The resulting pellets are resuspended in 200 μl of dissolution buffer (50 mM HEPES, 140 mM NaCl, 10 mM KCl, pH 7.2).

### Example 23

Crystallization and complexation of Etanercept (Enbrel). Entanercept (Enbrel) (human recombinant CHO cell derived) is desalted in 10 mM Tris buffer (pH 8.0) and concentrated to 30 mg/ml. One 0.5 ml portion of the antibody is then mixed with three portions of a crystallizing reagent containing 16% PEG-4000, 200 mM magnesium chloride and 100 mM Tris buffer at pH 8.6. The mixture is incubated overnight at room temperature. Entanercept (Enbrel) crystals are obtained the following day. After crystallization is complete, the crystals are washed with buffer (20% PEG-6000, 0.1 M Tris, pH 8.6). A 200 μl aliquot of crystals is removed and washed with buffer containing (20% PEG-6000 and 100 mM Tris buffer (pH 8.6).

To 13 μl of a 30 mg/ml suspension of crystals, polyarginine (10 μl 20 mg/ml, MW 90 kD) or polylysine (amount 10 μl, 20 mg/ml, MW, 90 kD) is added. The mixture is incubated at room temperature for 17 hours. After that, the complex is microcentrifuged for 5 minutes and the supernatant removed. The resulting pellets are resuspended in 200 μl of dissolution buffer (50 mM HEPES, 140 mM NaCl, 10 mM KCl, pH 7.2).

### Example 24

Crystallization of hGH with sodium acetate and 6% PEG-6000. Commercially available hGH was purified and/or dialyzed and concentrated as described in Example 1. Deionized water was added to the concentrated hGH solution to yield a final protein concentration of 15 mg/ml. Tris-HCl (1 M, pH 8.6) was added to a final concentration of 100 mM. To this solution, 6% (v/v) PEG-6000 was added. Crystals of hGH were grown by adding sodium acetate (Na-Acetate) (2 M) to the solution so that a final concentration of 500 mM Na-Acetate was obtained. The solution was then incubated for 16 hours at 25° C. Needle-like crystals were obtained and imaged by optical microscopy. The crystals obtained were found to be between about 25 and about 75 μm in length with a crystallization yield of greater than 85%.

### Example 25

Crystallization of hGH with sodium acetate. Here, a frozen bulk feed solution of soluble recombinantly-produced hGH (rhGH) was obtained from two stocks—one derived from E. coli (Novartis) and the other from yeast (Lucky Gold). Separate analyses of rhGH derived from E. coli and yeast stock solutions resulted in rhGH having the same crystallization and solubility characteristics irrespective of its source. Approximately 3.3 ml (10-20 mg/ml rhGH as supplied in unknown buffer) of thawed rhGH feed solution was purified using a 10DG-desalting column supplied by BioRad. Prior to sample loading, the column was conditioned by washing the column with 30 ml of Tris-HCl (10 mM, pH 8.0). The rhGH sample was then loaded and allowed to enter the column by gravity. After discarding the first three ml of eluant, another 5.0 ml of 10 mM Tris-HCl pH 8.0 was then added. 4.5 ml of the desalted rhGH was eluted and collected. Concentration by centrifugation was then performed using a Millipore concentrator (MWCO 10,000) at 3500 rpm for 20-30 min. The concentration of hGH was in range of 30 mg/ml as measured by absorbance at 280 nm/0.813 (1 mg/ml hGH A280=0.813 absorbance units). Crystals were grown by adding deionized water, Tris-HCl (pH 8.6), PEG-6000 and Na-acetate to final concentrations of 100 mM, 6% (v/v) and 500 mM, respectively in the total solution with a final protein concentration of 15 mg/ml. The solution was then mixed gently and incubated at 33° C. for 12-16 hours. Needle- or rod-like crystals were obtained and imaged with TEM (FIGS. 18A and 18B). The crystals ranged in length from approximately 2 to 25 μm. After centrifuging and pelleting the crystals the supernatant was extracted and, crystallization yield was measured as greater than 85%. The crystals can also be formed at temperatures between 33° C. and 15° C. but require increased crystallization time and possibly result in reduced yield.

Complexation of sodium hGH crystals with ionic polymer additive. Once crystallization yield was determined, sodium rhGH crystals were re-suspended in mother liquor (250 mM NaOAc, 25 mM Tris-HCl (pH 8.6), 6% PEG-6000, and either 7 mg/ml Protamine sulfate or 4.2 mg/ml polyarginine) so that a final concentration of 21 mg/ml of sodium rhGH crystals was achieved. The protein to additive ratio for rhGH to Protamine sulfate was approximately 3:1 (mg:mg) and for rhGH to Polyarginine was 5:1 (mg:mg). These ratios are calculated to be mole ratios of approximately 1:1.715 for rhGH:Protamine and approximately 1:0.587 for rhGH:polyarginine. The above rhGH pellets were homogenously re-suspended in the appropriate mother liquor and incubated overnight at 2-8° C. before being centrifuged to obtain a condensed pellet. The supernatants were removed and the pellets were re-suspended in the same mother liquor (without ionic polymer additive) and stored at 4° C.

Additional rhGH: ionic polymer additive ratios may be obtained by varying the additive concentration (mg/ml) of the mother liquor while still resuspending to 21 mg/ml of rhGH. For example, increased concentrations of Protamine Sulfate (10.5 mg/ml) in the mother liquor can be used to obtain a ratio upon resuspension of rhGH: additive of 2:1.

### Example 26

Comparative pharmacodynamic studies in female juvenile cynomologous monkeys. The goal of this study was to assess the in vivo pharmacokinetic profile of crystalline recombinant human growth hormone (rhGH) when administered subcutaneously to female Cynomologous monkeys. These data were generated in order to establish a model for controlled release of crystalline rhGH in blood serum and for weight gain as a function of crystalline rhGH release.

TABLE 4
Experimental Design for Primate Studies I

| | | Administration | | Dose | Dose | |
|---|---|---|---|---|---|---|
| | | of Dose^{c} | Dose Level | Concentration | Volume | Number of Animals |
| Group # | Sample | (hour) | (mg/kg) | mg/ml) | (ml/kg) | (Female) |
| 1 | Daily | 0, 24, 48, 72, | 0.8 | 3.2 | 0.25 | 4 |
| | Soluble^{a} | 96, 120, 144 | | | | |
| 2 | Na-Acetate, | 0 | 5.6 | 22.4 | 0.25 | 4 |
| | PEG, | | | | | |
| | polyarginine^{b} | | | | | |
| 3 | Na-Acetate, | 0 | 5.6 | 22.4 | 0.25 | 4 |
| | PEG, | | | | | |
| | protamine^{b} | | | | | |

^{a}Commercially-available hGH (soluble, uncrystallized form) was obtained from Novartis and diafiltered in WFI. Group 1 (positive control) received soluble hGH on each of the administration days.
^{b}See Example 25 for preparation.
^{c}All doses were delivered after daily bleed.

Twelve female juvenile cynomologous monkeys were divided into three groups, each having four animals per group, and were administered either soluble rhGH (Group 1), sodium crystals of rhGH with PEG and polyarginine (Group 2, according to Example 25) or sodium crystals of rhGH with PEG and protamine (Group 3, according to Example 25). The monkeys, ranging from 2-6 kg in weight and 4-7 years of age at the onset of treatment, were individually housed in stainless steel cages equipped with an automatic watering system or water bottles. The animal room environment was controlled (approximately 21±3° C., 30-70% humidity, 12 hours light and 12 hours darkness in each 24-hour period, and 12-20 air changes per hour) and twice daily, the monkeys were fed a standard certified commercial primate chow (Harlan Teklad Certified Primate Diet #2055C).

This primate study was conducted in order to measure and compare serum concentrations of hGH and Insulin-Like Growth Factor (IGF-1) after the administration of soluble rhGH (Group 1), sodium crystals of rhGH with PEG and polyarginine (Group 2) and sodium crystals of rhGH with PEG and protamine (Group 3). Body weights were recorded for all animals at transfer and prior to dosing on the times indicated in Table 5 above. Blood samples (approximately 1 ml) were collected from each animal via the femoral, brachial or saphenous vein on the mornings of days −216, −120, 0, 2, 4, 6, 8, 10, 24, 48, 72, 96, 120, 144, 168, 192, 216, 240, 264, 288 and 312. Blood was collected into serum separating tubes, left at room temperature for 30-45 minutes to allow clotting, and centrifuged at 2-8° C. for 10 minutes at 3000 rpm. Each serum sample was split into a 100 μl aliquot and remaining aliquot, both of which were stored at −70±10° C. prior to analysis. Typically, the smaller 100 μl aliquot was used for rhGH determination and the larger remainder was used for IGF-1 determination. There were some exceptions, due to volume of replicates needed.

Collected serum samples were then analyzed for hGH concentration (see Table 5). Note that appropriate dilutions were made to rhGH concentrations that fell outside the standard value range. All values were used to obtain an individual per animal average background level of primate hGH. This per animal average was subtracted from the serum levels measured at each time point for that test subject. The corrected values per time point were then averaged to obtain a corrected mean of rhGH in serum. Standard errors were then calculated by using standard deviation of the corrected mean and divided by the square root of N=4.

TABLE 5
rhGH levels for groups 1 (daily soluble), 2
(sodium rhGH/polyarginine) and 3 (sodium rhGH/protamine)

| | Group 1 - | | Group 2 - | | Group 3 - | |
|---|---|---|---|---|---|---|
| | Average | | Average | | Average | |
| | daily | | sodium | | sodium | |
| Time | soluble | | rhGH/ | | rhGH/ | |
| in | rhGH | Std. | polyarginine, | Std. | protamine, | Std. |
| hrs | (ng/ml) | Err. | (ng/ml) | Err. | (ng/ml) | Err. |
| −216 | 4 | 7 | −4 | 7 | −2 | 1 |
| −120 | 8 | 9 | 7 | 5 | 7 | 9 |
| 0 | 343 | 65 | −4 | 3 | −5 | 9 |
| 2 | 372 | 48 | −6 | 6 | 1 | 13 |
| 4 | 262 | 37 | 11 | 9 | 20 | 12 |
| 6 | 205 | 45 | 85 | 45 | 94 | 35 |
| 8 | 132 | 29 | 186 | 93 | 159 | 57 |
| 10 | 18 | 37 | 409 | 202 | 381 | 189 |
| 24 | −14 | 7 | 404 | 17 | 333 | 37 |
| 48 | −7 | 8 | 178 | 33 | 216 | 43 |
| 72 | −3 | 10 | 77 | 35 | 86 | 18 |
| 96 | −9 | 9 | 12 | 14 | 21 | 13 |
| 120 | −11 | 6 | 6 | 13 | 2 | 11 |
| 144 | −3 | 13 | −6 | 10 | 3 | 13 |
| 168 | 10 | 11 | −2 | 4 | −1 | 11 |
| 192 | −11 | 10 | 3 | 2 | 0 | 7 |
| 216 | −13 | 9 | 18 | 12 | 9 | 6 |
| 240 | 1 | 5 | 18 | 14 | 31 | 12 |
| 264 | 8 | 3 | 20 | 5 | 17 | 11 |
| 288 | −15 | 8 | 1 | 4 | 17 | 11 |
| 312 | 4 | 7 | 1 | 5 | 8 | 17 |

Note:
rhGH value is the average value from 4 animals that has been baseline adjusted, i.e., value mimus baselime. Baseline is the average of values at t = −216, −120 and 0 hours.

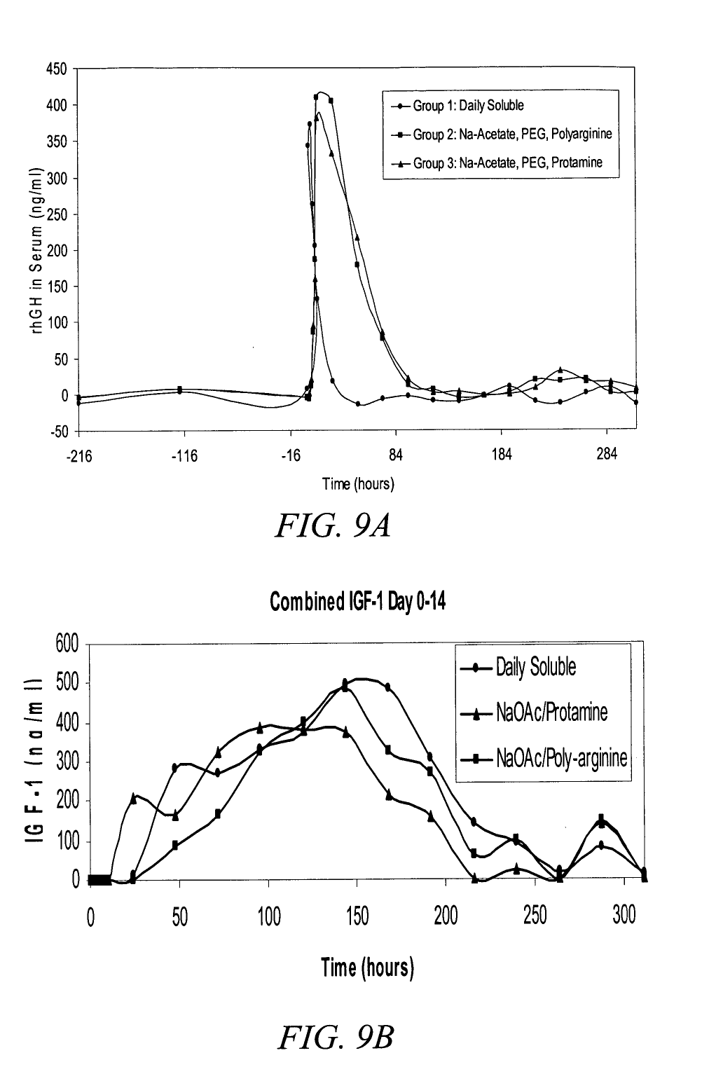

FIG. 9A illustrates the level of rhGH in serum, after baseline adjustment, as a function of time in hours for Groups 1, 2 and 3.

TABLE 6
Summary of pharmacokinetic parameters based on
data in Table 5

| | Group 1^{a} | Group 2 | Group 3 |
|---|---|---|---|
| Dose Amount (mg) | 3.2 | 22.4 | 22.4 |
| Dosage (mg/kg) | 0.8 | 5.6 | 5.6 |
| Cₘₐₓ (ng/ml) | 372 | 409 | 381 |
| Tₘₐₓ (hr) | 2 | 10 | 10 |
| AUC (0 − t) | 4570 | 3503 | 3455 |
| (ng. hr. kg/ml. mg) | | | |
| T_{90%} (hr) | 20 | 74 | 77 |

^{a}Commercially-available hGH (soluble, uncrystallized form) was diafiltered in WFI. Group 1 (positive control) received soluble hGH on each of the seven administration days.

The data above demonstrates that the time at which maximum hGH appeared in the serum (Tₘₐₓ) was 10 hours for the polyarginine complexed sodium crystal hGH, 10 hours for the Protamine complexed sodium crystal hGH and 2 hours for the soluble hGH. Even though the soluble hGH was delivered at 1/7th the dose of the crystal administrations, the Cₘₐₓ values listed above in Table 6 show that hGH when delivered in either of the complexed crystalline forms significantly reduce the initial serum concentration spike. In addition, a T_{90%} value has been calculated for the soluble and crystalline groups. The T_{90%} for Group 1, the soluble form, was 20 hours, whereas the T_{90%} for Groups 2 and 3, the complexed crystalline forms, were 74 and 77 hours, respectively. These results clearly show that the complexed crystalline forms result in elevated hGH levels for significantly longer times than that of the soluble form.

In addition to determination of serum concentrations of hGH, the level of IGF-1 was also measured as a function of time. By measuring the production of IGF-1, the efficacy of rhGH was ascertained. Table 7 below reports the IGF-1 concentrations for animals in Groups 1-3. FIG. 9B illustrates that following baseline subtraction of endogenous IGF-1 levels, complexed crystalline formulations have demonstrated the ability to stimulate IGF-1 release comparable to daily soluble administrations. These results, in non-human primates, indicate that formulations according to this invention may be advantageously used to achieve similar efficacy in humans.

TABLE 7
IGF-1 levels for Group, Daily soluble, sodium
rhGH/polyarginine and Sodium rhGH/protamine

| | | | | | Group 3 - | |
|---|---|---|---|---|---|---|
| | Group 1 - | | Group 2 - | | Average | |
| | Average | | Average | | sodium | |
| | daily | | sodium | | rhGH/ | |
| Time | soluble | | rhGH/ | | protamine, | |
| in | IGF-1 | Std. | polyarginine, | Std. | IGF-1 | Std. |
| hrs | (ng/ml) | Err. | IGF-1 (ng/ml) | Err. | (ng/ml) | Err. |
| −216 | −48 | 54 | 263 | 148 | 31 | 94 |
| −120 | −0.63 | 22 | 4 | 34 | 218 | 115 |
| 0 | 48 | 73 | −268 | 158 | −249 | 86 |
| 2 | 39 | 32 | −160 | 146 | −56 | 104 |
| 4 | 22 | 52 | −344 | 191 | −120 | 114 |
| 6 | 37 | 45 | −244 | 189 | −19 | 96 |
| 8 | −22 | 9 | −464 | 170 | −66 | 119 |
| 10 | 106 | 63 | −491 | 219 | 4 | 86 |
| 24 | 130 | 130 | −106 | 278 | 223 | 214 |
| 48 | 446 | 59 | 164 | 244 | 191 | 164 |
| 72 | 414 | 95 | 248 | 224 | 340 | 207 |
| 96 | 485 | 114 | 402 | 67 | 416 | 243 |
| 120 | 524 | 73 | 484 | 126 | 392 | 216 |
| 144 | 636 | 63 | 574 | 189 | 397 | 187 |
| 168 | 636 | 82 | 415 | 191 | 240 | 176 |
| 192 | 438 | 71 | 356 | 136 | 227 | 153 |
| 216 | 288 | 87 | 155 | 108 | 117 | 146 |
| 240 | 210 | 69 | 197 | 57 | 93 | 144 |
| 264 | 161 | 67 | 88 | 82 | 85 | 167 |
| 288 | 222 | 113 | 243 | 95 | 201 | 208 |
| 312 | 178 | 175 | 79 | 120 | 86 | 149 |

Note:
IGF-1 values are reported as the average values calculated from 4 animals that have been baseline adjusted, i.e., value minus baseline. Baseline is the average of values at t = −216, −120 and 0 hours.

### Example 27

Pharmcocodynamic study of human growth hormone administered by single or daily subcutaneous injection to hypophysectomized male rats. The goal of this study was to compare the efficacy of different formulations of hGH when administered once or daily for seven consecutive days subcutaneously to hypophysectomized male Wistar rats. The study design was as follows:

TABLE 8
Study Design - Sample Description

| Group # | | |
|---|---|---|
| or Test | | |
| Compound | Sample^{a} | Sample Description |
| 1 | Daily Soluble | (no hGH) 16.7 mg/ml D-mannitol, 26.7 mg/ml |
| | Vehicle - Sham | sucrose, 50 mM NaH₂PO₄ (pH 6.5) |
| | Hypophysectomy | |
| 2 | Daily Soluble | (no hGH) 16.7 mg/ml D-mannitol, 26.7 mg/ml |
| | Vehicle - Low | sucrose, 50 mM NaH₂PO₄ (pH 6.5) |
| | Dose | |
| 3 | Daily Soluble | (no hGH) 16.7 mg/ml D-mannitol, 26.7 mg/ml |
| | Vehicle - High | sucrose, 50 mM NaH₂PO₄ (pH 6.5) |
| | Dose | |
| 4 | Daily Soluble - | 0.71 mg/ml rhGH, 16.7 mg/ml D-mannitol, |
| | Low Dose | 26.7 mg/ml sucrose, 50 mM NaH₂PO₄ (pH 6.5) |
| | Low Dose | |
| 5 | Daily Soluble | 1.0 mg/ml rhGH, 16.7 mg/ml D-mannitol, |
| | Dose | 26.7 mg/ml sucrose, 50 mM NaH₂PO₄ (pH 6.5) |
| 6 | Soluble - | 3.5 mg/ml rhGH, 16.7 mg/ml D-mannitol, |
| | Single Bolus | 26.7 mg/ml sucrose, 50 mM NaH₂PO₄ (pH 6.5) |
| | High Dose | |
| 7 | Polyarginine | 18.7 mg/ml crystalline rHGH, 250 mM NaOAc, |
| | Crystals - High | 6% PEG-6000, 25 mM Tris-HCl (pH 8.6), 3.6 |
| | Dose | mg/ml polyarginine HCl (molar ratio of |
| | | rhGH:polyarginine is 1:0.587 |
| 8 | Soluble - | 250 mM NaOAc, 6% PEG-6000, 25 mM Tris-HCl |
| | Protamine | (pH 8.6), 0.75 mg/ml protamine sulfate |
| | Crystals | |
| 9 | Protamine | 3.3 mg/ml crystalline rHGH, 250 mM NaOAc, |
| | Crystals - Low | 6% PEG-6000, 25 mM Tris-HCl (pH 8.6), 0.75 |
| | Dose | mg/ml protamine sulfate (molar ratio of |
| | | rhGH:polyarginine is 1:1.715 |
| 10 | Protamine | 18.7 mg/ml crystalline rHGH, 250 mM NaOAc, |
| | Crystals - High | 6% PEG-6000, 25 mM Tris-HCl (pH 8.6), 4 |
| | Dose | mg/ml protamine sulfate (molar ratio of |
| | | rhGH:polyarginine is 1:1.715. |
| 11 | Vehicle | 250 mM NaOAc, 6% PEG-6000, 25 mM Tris-HCl |
| | Control- | (pH 8.6), 3.6 mg/ml polyarginine-HCl |
| | Polyarginine | |
| | Crystals | |
| 12 | Vehicle | 16.7 mg/ml D-mannitol, 26.7 mg/ml sucrose, |
| | Control- Single | 50 mM NaH₂PO₄ (pH 6.5) |
| | Bolus | |

^{a}All samples were prepared using WFI under sterile conditions. The vehicle and soluble hGH samples were filtered with 0.22 μm filter after bringing the solutions to their respective final volumes.

TABLE 9
Study Design - Administration

| | | | | | Number |
|---|---|---|---|---|---|
| Group # | | | Dose | | of |
| or Test | Dose Level | Dose Conc. | volume | Dose | Animals |
| Compound | (mg/kg) | (mg/ml) | (μl) | Regimen | (males) |
| 1 | | 0 | 200 | 7 daily | 13 |
| | | | | doses | |
| 2 | 0 | 0 | 20 | 7 daily | 11 |
| | | | | doses | |
| 3 | 0 | 0 | 80 | 7 daily | 11 |
| | | | | doses | |
| 4 | 0.143 | 0.71 | 20 | 7 daily | 11 |
| | | | | doses | |
| 5 | 0.8 | 1 | 80 | 7 daily | 12 |
| | | | | doses | |
| 6 | 5.6 | 3.5 | 160 | Day 1 | 11 |
| 7 | 5.6 | 18.7 | 30 | Day 1 | 12 |
| 8 | 0 | 0 | 30 | Day 1 | 11 |
| 9 | 1 | 3.3 | 30 | Day 1 | 12 |
| 10 | 5.6 | 18.7 | 30 | Day 1 | 12 |
| 11 | 0 | 0 | 30 | Day 1 | 11 |
| 12 | 0 | 0 | 30 | Day 1 | 11 |

Upon arrival, 138 male Wistar rats, weighing approximately 90-100 grams and being approximately 25-30 days old, were group-housed under controlled conditions (approximate temperature 23±3° C., relative humidity 30-70%, 12 hours light and 12 hours darkness in each 24-hour period, 10-15 air changes per hour) and given access to purified water and laboratory chow ad libitum throughout the study. The rats were allowed to acclimate to the environment for two weeks prior to testing.

The 138 rats were administered samples according to the concentration, volume and dosing regimen in Table 9. The test compounds were administered once or once daily for seven consecutive days as a single bolus injection subcutaneously in the dorsum area. The site of injection was shaved and marked up to 3 days prior to dosing and thereafter as required to facilitate injection. The test compounds were administered using a 30-gauge×8 mm needle attached to a 300 μl syringe. Test compounds were carefully inverted in order to ensure suspension or solution uniformity without causing foaming prior to withdrawal into the syringe and again prior to administration.

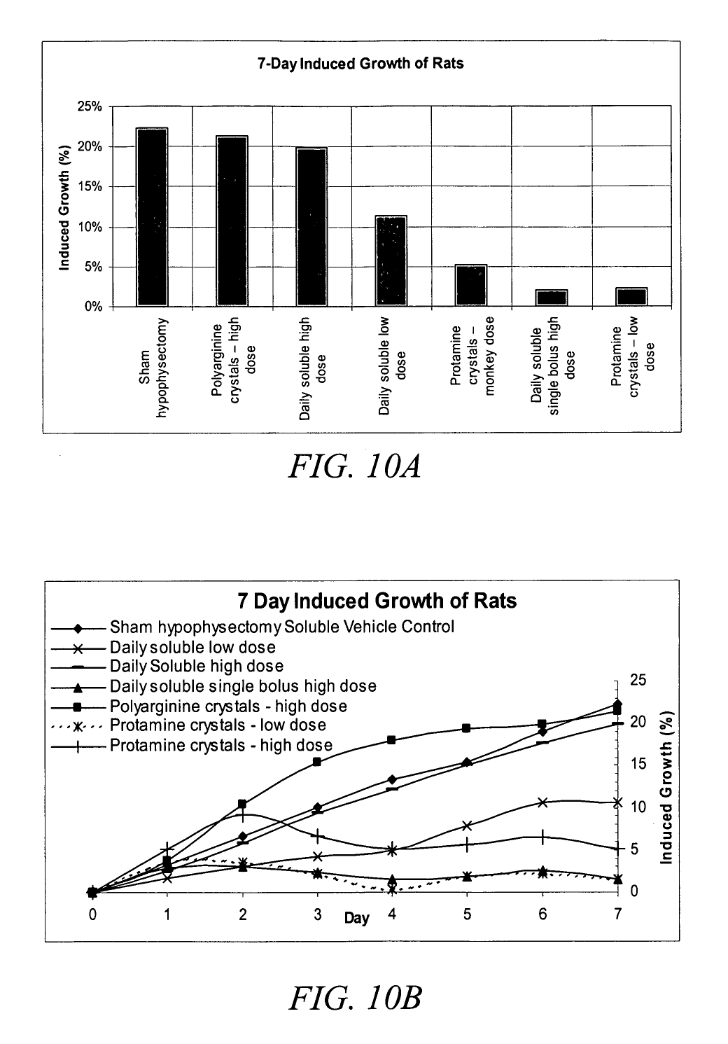

Weight gain was measured and recorded twice weekly during weeks −3 and −2 and daily from days −7 through 14. Rat weights were approximately 100 g±10% at dosing. The results of percent induced growth are presented in FIGS. 10A and 10B and summarized in Tables 10 and 11. In Table 10, “high dose” represents 5.6 mg/kg/week. The data illustrates the comparison of the weight gain of rats having a single injection of rhGH:polyarginine (Group 7, Example 25) or rhGH:protamine (Groups 9 and 10, Example 25) crystals over a seven day period versus a daily injection of control (Group 1, no hGH) or soluble hGH samples (Groups 4 and 5) over the same seven day period. Group 1, Sham Hypophysectomy rats, shows the normal growth over a seven day period. Moreover, rats having been administered rhGH:polyarginine (Group 7) had a higher percent induced growth with one injection over seven days than those rats that were administered soluble hGH (Group 5) each day for seven days. Observed differences between daily soluble injections and single injections of polyarginine complexed crystalline rhGH cannot be statistically verified. These results illustrate that hGH crystals and formulations according to the present invention are as efficacious as daily soluble rhGH administered over one week.

TABLE 10
8 Day Induced Weight Gain in Hypophysectomized Rats

| Group # | | Day 8 |
|---|---|---|
| or Test | | Induced |
| Article | Sample Description | Growth |
| 1 | Sham hypophysectomy | 22% |
| 7 | Polyarginine crystals - | 21% |
| | high dose | |
| 5 | Daily Soluble high dose | 20% |
| 4 | Daily soluble low dose | 11% |
| 10 | Protamine crystals - high | 5% |
| | dose | |
| 6 | Daily soluble single bolus | 2% |
| | high dose | |
| 9 | Protamine crystals - low | 2% |
| | dose | |

TABLE 11
Daily Induced Weight Gain (grams)
in Hypophysectomized Rats

| | Day | | | | | | | |
|---|---|---|---|---|---|---|---|---|
| Group | 0 | 1 | 2 | 3 | 4 | 5 | 6 | 7 |
| 1 | 0 | 3 | 7 | 10 | 13 | 15 | 19 | 22 |
| 7 | 0 | 4 | 10 | 15 | 18 | 19 | 20 | 21 |
| 4 | 0 | 2 | 3 | 4 | 5 | 8 | 11 | 11 |
| 5 | 0 | 3 | 6 | 10 | 12 | 15 | 18 | 20 |
| 10 | 0 | 5 | 9 | 7 | 5 | 6 | 6 | 5 |
| 6 | 0 | 3 | 3 | 2 | 2 | 2 | 3 | 2 |
| 9 | 0 | 4 | 4 | 2 | 0 | 2 | 2 | 2 |

### Example 28

Crystallization of hGH with sodium acetate and Protamine sulfate. Here, a frozen bulk feed solution of soluble recombinantly-produced hGH (rhGH) was obtained from two stocks—one derived from E. coli (Novartis) and the other from yeast (Lucky Gold). Separate analyses of rhGH derived from E. coli and yeast stock solutions resulted in rhGH having the same crystallization and solubility characteristics irrespective of its source. Approximately 3.3 ml (10-20 mg/ml) of thawed rhGH feed solution was purified using a 10DG-desalting column supplied by BioRad. Prior to sample loading, the column was conditioned by washing the column with 30 ml of Tris-HCl (10 mM, pH 8.0). The rhGH sample was then loaded and allowed to enter the column by gravity. After discarding the first three ml of eluant, another 5.0 ml of 10 mM Tris-HCl pH 8.0 was added. 4.5 ml of the desalted rhGH was eluted and collected. Concentration by centrifugation was then performed using a Millipore concentrator (MWCO 10,000) at 3500 rpm for 20-30 min. The concentration of hGH was in range of 30 mg/ml as measured by absorbance at 280 nm/0.813 (1 mg/ml hGH A280=0.813 absorbance units). Crystals were grown by adding deionized water, Tris-HCl (pH 8.6), PEG-4000, Protamine sulfate and Na-acetate to final concentrations of 100 mM, 6% (v/v), 2 mg/ml and 500 mM, respectively, in the total solution with a final protein concentration of 15 mg/ml. The solution was then mixed gently and incubated at 33° C. for 12-16 hours. Needle-like crystals were obtained ranging in length from approximately 2 to 25 μm. After centrifuging and pelleting the crystals the supernatant was extracted and, crystallization yield was measured as greater than 90%.

### Example 29

Crystallization of hGH with sodium acetate and polyarginine HCl. Here, a frozen bulk feed solution of soluble recombinantly-produced hGH (rhGH) was obtained from two stocks—one derived from E. coli (Novartis) and the other from yeast (Lucky Gold). Separate analyses of rhGH derived from E. coli and yeast stock solutions resulted in rhGH having the same crystallization and solubility characteristics irrespective of its source. Approximately 3.3 ml (10-20 mg/ml) of thawed rhGH feed solution was purified using a 10DG-desalting column supplied by BioRad. Prior to sample loading, the column was conditioned by washing the column with 30 ml of Tris-HCl (10 mM, pH 8.0). The rhGH sample was then loaded and allowed to enter the column by gravity. After discarding the first three ml of eluant, another 5.0 ml of 10 mM Tris-HCl pH 8.0 was added. 4.5 ml of the desalted rhGH was eluted and collected. Concentration by centrifugation was then performed using a Millipore concentrator (MWCO 10,000) at 3500 rpm for 20-30 min. The concentration of hGH was in range of 30 mg/ml as measured by absorbance at 280 nm/0.813 (1 mg/ml hGH A280=0.813 absorbance units). Crystals were grown by adding deionized water, Tris-HCl (pH 8.6), PEG-4000, polyarginine HCl and Na-acetate to final concentrations of 100 mM, 2% (v/v), 2 mg/ml and 500 mM, respectively, in the total solution with a final protein concentration of 15 mg/ml. The solution was then mixed gently and incubated at 33° C. for 12-16 hours. Needle-like crystals were obtained ranging in length from approximately 2 to 25 μm. After centrifuging and pelleting the crystals the supernatant was extracted and, crystallization yield was measured as greater than 90%.

Although the foregoing invention has been described in some detail by way of illustration and example for purposes of clarity of understanding, it will be readily apparent to hose of ordinary skill in the art in light of the teachings of this invention that certain changes and modifications may be made thereto without departing from the spirit or scope of the disclosure herein, including the appended embodiments.

We claim:

1. A complex comprising a protein crystal and an ionic compound.

2. The complex according to claim 1, wherein said protein is selected from the group consisting of: therapeutic proteins, fusion proteins, glycoproteins, receptors, synthetic antigens, recombinant antigens, viral surface proteins, hormones, antibodies, enzymes, Fab fragments, cyclic peptides, linear peptides.

3. The complex according to claim 2, wherein said therapeutic protein is selected from the group consisting of: glucagon-like peptide 1, antibodies, histocompatibility antigens, integrins, selecting, inhibitors, growth factors, postridical hormones, nerve growth hormones, blood clotting factors, adhesion molecules, bone morphogenic proteins, lectins, trophic factors, cytokines such as TGF-β, IL-2, IL-4, α-IFN, β-IFN, γ-IFN, TNF, IL-6, IL-8, lymphotoxin, IL-5, Migration inhibition factor, GMCSF, IL-7, IL-3, monocyte-macrophage colony stimulating factors, granulocyte colony stimulating factors, multidrug resistance proteins, other lymphokines, toxoids, erythropoietin, Factor VIII, amylin, TPA, dornase-α, α-1-antitrypsin, human growth hormones, nerve growth hormones, bone morphogenic proteins, growth differentiation factors, neuregulin, urease and toxoids.

4. The complex according to claim 2, wherein said hormone is selected from the group consisting of: human growth hormone, glucagons, parathyroid hormone, fertility hormones, lutenizing hormone and follicle stimulating hormone.

5. The complex according to claim 2, wherein said antibody is selected from the group consisting of: Infliximab, Etanercept, Rituximab, trastuzumab, Abciximab, Palivizumab, Murumonab-CD3, Gemtuzumab, Basiliximab, Daclizumab, Zevalin and Mylotarg.

6. The complex according to claim 2, wherein said enzyme is selected from the group consisting of: rasburicase, lipase, amylase, hydrolases, oxidases, isomerases, lyases, ligases, adenylate cyclases, transferases, oxidoreductases, nitrilases, laccase, dehydrogenase, peroxidases and hydantoinase.

7. The complex according to claim 1, wherein said ionic compound is selected from the group consisting of: polymers, polypeptides, oligopeptides, proteins and dendrimers.

8. The complex according to claim 7, wherein said polypeptide or protein component of said ionic compound has a molecular weight of greater than about 2 kD.

9. The complex according to claim 7, wherein said polypeptide or said protein component of said ionic compound is selected from the group consisting of: polycations and polyanions.

10. The complex according to claim 9, wherein said polycation is selected from the group consisting of: Protamine, polyarginine, polylysine, polyhistidine, histones, myelinbasic protein, polymyxin B sulfate, dodecyltrimethylammonium bromide, bradykinin, spermine, putrescine, octylarginine and synthetic peptides and dendrimers.

11. The complex according to claim 9, wherein said polyanion is selected from the group consisting of: polyglutamate, polyaspartate, polyacrylate, polycyanoacrylates, polylactate, poly-B-hydroxybutyrate, polyvinylpyrollidone, hyaluronic acid, heparin, sulfated polysaccharides, dextran sulfates, heparin sulfates and dendrimers.

12. A composition comprising an insoluble phase suspended in a solution phase, wherein said insoluble phase is a complex comprising a protein crystal, an ionic compound and an excipient and wherein said solution phase is selected from the group consisting of: water, buffer, preservative, isotonicity agents, stabilizers and combinations thereof.

13. The composition according to claim 12, wherein said protein is selected from the group consisting of: therapeutic proteins, fusion proteins, glycoproteins, receptors, synthetic antigens, recombinant antigens, viral surface proteins, hormones, antibodies, enzymes, Fab fragments, cyclic peptides, linear peptides.

14. The composition according to claim 13, wherein said therapeutic protein is selected from the group consisting of: glucagon-like peptide 1, antibodies, histcompatibility antigens, integrins, selectins, inhibitors, growth factors, postridical hormones, nerve growth hormones, blood clotting factors, adhesion molecules, bone morphogenic proteins and lectins, trophic factors, cytokines such as TGF-β, IL-2, IL-4, α-IFN, β-IFN, γ-IFN, TNF, IL-6, IL-8, lymphotoxin, IL-5, Migration inhibition factor, GMCSF, IL-7, IL-3, monocyte-macrophage colony stimulating factors, granulocyte colony stimulating factors, multidrug resistance proteins, other lymphokines, toxoids, erythropoietin, Factor VIII, amylin, TPA, dornase-α, α-1-antitrypsin, human growth hormones, nerve growth hormones, bone morphogenic proteins, urease and toxoids.

15. The composition according to claim 13, wherein said hormone is selected from the group consisting of: human growth hormone, human growth hormone, glucagons, parathyroid hormone, fertility hormones, lutenizing hormone and follicle stimulating hormone.

16. The composition according to claim 13, wherein said antibody is selected from the group consisting of: Infliximab, Etanercept, Rituximab, trastuzumab, Abciximab, Palivizumab, Murumonab-CD3, Gemtuzumab, Basiliximab, Daclizumab, Zevalin and Mylotarg.

17. The composition according to claim 13, wherein said enzyme is selected from the group consisting of: rasburicase, lipase, amylase, hydrolases, oxidases, isomerases, lyases, ligases, adenylate cyclases, transferases, oxidoreductases, nitrilases, laccase, dehydrogenase, peroxidases and hydantoinase.

18. The composition according to claim 12, wherein said ionic compound is selected from the group consisting of: polymers, polypeptides, oligopeptides, proteins and dendrimers.

19. The composition according to claim 18, wherein said oligopeptide component of said ionic compound has a molecular weight of less than about 2 kD.

20. The composition according to claim 18, wherein said polypeptide or protein component of said ionic compound has a molecular weight of greater than about 2 kD.

21. The composition according to claim 18, wherein said polypeptide or said protein component of said ionic compound is selected from the group consisting of polycations and polyanions.

22. The composition according to claim 21, wherein said polycation is selected from the group consisting of: Protamine, polyarginine, polylysine, polyhistidine, histones, myelinbasic protein, polymyxin B sulfate, dodecyltrimethylammonium bromide, bradykinin, spermine, putrescine, octylarginine and synthetic peptides and dendrimers.

23. The composition according to claim 21, wherein said polyanion is selected from the group consisting of: polyglutamate, polyaspartate, polyacrylate, polycyanoacrylates, polylactate, poly-B-hydroxybutyrate, polyvinylpyrollidone, hyaluronic acid, heparin, sulfated polysaccharides, dextran sulfates, heparin sulfates and dendrimers.

24. The composition according to claim 12, wherein said excipient is selected from the group consisting of: detergents, pluronic polyols, polyols, glycoaminoglycans, amino acids, starch, glycerol, sugars, cellulose, povidone dextrin, polysorbates, hydroxypropyl cellulose and ascorbic acid.

25. The composition according to claim 12, wherein said stabilizer is selected from the group consisting of: sugars, polyols, amino acids, soluble proteins and detergents.

26. A method for treating a disease state in a mammal, comprising the step of administering to said mammal a therapeutically effective amount of a complex according to any one of claims 1-11.

27. A method for treating a disease state in a mammal, comprising the step of administering to said mammal a therapeutically effective amount of a composition according to any one of claims 12-25.

28. The method according to claim 26 or 27, wherein said complex or composition is administered to said mammal by oral route or parenteral route.

29. The method according to claim 28, wherein said complex or composition is administered to said mammal by subcutaneous or intramuscular route.

30. The method according to claim 29, wherein said complex or composition is administered to said mammal by subcutaneous route using a needle having a gauge greater than 27.

31. The method according to claim 26 or 27, wherein said complex or composition is administered to said mammal by needle-free injection or by transdermal means.

32. The method according to claim 26 or 27, wherein said complex or composition is administered to said mammal once a week.

33. The method according to claim 26 or 27, wherein said complex or composition is administered to said mammal once every two weeks.

34. The method according to claim 26 or 27, wherein said complex or composition is administered to said mammal once a month.

35. The method according to claim 26 or 27, wherein said mammal is a human.

36. A method for producing a protein complex, comprising the steps of:
(a) mixing a solution of a protein with a crystallization reagent mix to produce a solution;
(b) adding deionized water to said solution;
(c) incubating said solution for between about 2 and about 48 hours at a temperature between about 4° C. and about 40° C., until protein crystals are formed; and
(d) adding an ionic compound to said solution.

37. A method for producing a protein complex, comprising the steps of:
(a) mixing a solution of a protein with a crystallization buffer to produce a solution;
(b) adding deionized water to said solution;
(c) adding an ionic compound to said solution; and
(d) incubating said solution for between about 2 and about 48 hours at a temperature between about 4° C. and about 40° C., until protein crystals are formed.

38. The method according to claim 35 or 36, which further comprises the step of adding an excipient to said solution between steps (b) and (c).

39. A method for producing a composition comprising a protein complex suspended in a solution phase, comprising the step of mixing said complex prepared according to claim 35 or 36 in a solution phase selected from the group consisting of: water, buffer, preservative, isotonicity agents, stabilizers and combinations thereof.

40. The method according to claim 35 or 36, wherein, in step (a), said protein is present in said solution at a concentration between about 0.5 mg/ml and about 200 mg/ml.

41. The method according to claim 35 or 36, wherein, in step (a), said crystallization reagent mix is selected from the group consisting of: Tris-HCl, HEPES, acetate, phosphate, citrate borate, imidazole, Bis-tris, bicarbonate, carbonate, N-(2-acetamido)-iminodiacetic acid and MES.

42. The method according to claim 35 or 36, wherein said crystallization reagent mix is present in said solution at a concentration between about 0.5 mM and about 500 mM.

43. The method according to claim 35 or 36, wherein said crystallization reagent mix has a pH between about 2 and about 10.

44. The method according to claim 35 or 36, wherein in step (d), the pH of said solution is the same as the pH of said crystallization reagent mix.

45. The method according to claim 35 or 36, wherein, in step (c) of claim 35 and in step (d) of claim 36, said solution is incubated for between about one and about two days at a temperature between about 4° C. and about 37° C.

46. The method according to claim 35 or 36, wherein said ionic compound is selected from the group consisting of: polymers, polypeptides, oligopeptides, proteins and dendrimers.

47. The method according to claim 45, wherein said oligopeptide component of said ionic compound has a molecular weight of less than about 2 kD.

48. The method according to claim 45, wherein said polypeptide or protein component of said ionic compound has a molecular weight of greater than about 2 kD.

49. The method according to claim 45, wherein said polypeptide or said protein component of said ionic compound is selected from the group consisting of polycations and polyanions.

50. The method according to claim 48, wherein said polycation is selected from the group consisting of: Protamine, polyarginine, polylysine, polyhistidine, histones, myelinbasic protein, polymyxin B sulfate, dodecyltrimethylammonium bromide, bradykinin, spermine, putrescine, octylarginine and synthetic peptides and dendrimers.

51. The method according to claim 48, wherein said polyanion is selected from polyglutamate, polyaspartate, polyacrylate, polycyanoacrylates, polylactate, poly-B-hydroxybutyrate, polyvinylpyrollidone, hyaluronic acid, heparin, sulfated polysaccharides, dextran sulfates, heparin sulfates and dendrimerspolyglutamate and polyaspartate.

52. The method according to claim 37, wherein said excipient is selected from the group consisting of: detergents, pluronic polyols, polyols, glycoaminoglycans, amino acids, starch, glycerol, sugars, cellulose, povidone dextrin, polysotbates, hydroxypropyl cellulose and ascorbic acid.

53. The method according to claim 38, wherein said stabilizer is selected from the group consisting of: sugars, polyols, amino acids, soluble proteins, detergents and combinations thereof.

54. The method according to claim 35 or 36, wherein said protein is selected from the group consisting of: therapeutic proteins, fusion proteins, glycoproteins, receptors, synthetic antigens, recombinant antigens, viral surface proteins, hormones, antibodies, enzymes, Fab fragments, cyclic peptides, linear peptides.

55. The method according to claim 35 or 36, wherein said therapeutic protein is selected from the group consisting of: glucagon-like peptide 1, antibodies, histocompatibility antigens, integrins, selectins, inhibitors, growth factors, postridical hormones, nerve growth hormones, blood clotting factors, adhesion molecules, bone morphogenic proteins, lectins, trophic factors, cytokines such as TGF-β, IL-2, IL-4, α-IFN, β-IFN, γ-IFN, TNF, IL-6, IL-8, lymphotoxin, IL-5, Migration inhibition factor, GMCSF, IL-7, IL-3, monocyte-macrophage colony stimulating factors, granulocyte colony stimulating factors, multidrug resistance proteins, other lymphokines, toxoids, erythropoietin, Factor VIII, amylin, TPA, dornase-α, α-1-antitrypsin, human growth hormones, nerve growth hormones, bone morphogenic proteins, growth differentiation factors, reuregulins, urease and toxoids.

56. The method according to claim 35 or 36, wherein said hormone is selected from the group consisting of: human growth hormone, glucagons, parathyroid hormone, fertility hormones, lutenizing hormone and follicle stimulating hormone.

57. The method according to claim 35 or 36, wherein said antibody is selected from the group consisting of: Infliximab, Etanercept, Rituximab, trastuzumab, Abciximab, Palivizumab, Murumonab-CD3, Gemtuzumab, Basiliximab, Daclizumab, Zevalin and Mylotarg.

58. The method according to claim 35 or 36, wherein said enzyme is selected from the group consisting of: rasburicase, lipase, amylase, hydrolases, oxidases, isomerases, lyases, ligases, adenylate cyclases, transferases, oxidoreductases, nitrilases, laccase, dehydrogenase, peroxidases and hydantoinase.

59. The method according to claim 35 or 36, wherein said enzyme is selected from the group consisting of: Aspergillus oryzae amylase, Burkholderia cepacia lipase, oxalate oxidase and urate oxidase.

60. The complex according to claim 1 or 2, wherein said protein crystal is not an insulin protein crystal.
